SEQUENCE LISTING (1 sequence)

SEQ ID NO: 1 - protein, 473 aa, Artificial sequence
  Amino acid sequence of CAR
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0148586 A1
(43) Pub. Date: May 18, 2023

(54) LOW-TEMPERATURE STORAGE OF BIOLOGICAL SAMPLES

(71) Applicant: CRAGE MEDICAL CO., LIMITED, Hong Kong (CN)

(72) Inventors: Zonghai LI, Shanghai (CN); Huamao WANG, Shanghai (CN); Huiping GAO, Shanghai (CN)

(73) Assignee: CRAGE MEDICAL CO., LIMITED, Hong Kong (CN)

(21) Appl. No.: 17/920,515

(22) PCT Filed: Apr. 21, 2021
(86) PCT No.: PCT/CN2021/088745

§ 371 Date: Oct. 21, 2022

(30) Foreign Application Priority Data
Apr. 21, 2020 (CN) 202010316560.3

(51) Int. Cl. A01N 1/02; C12N 5/078

(52) U.S. Cl. CPC A01N 1/0221; C12N 5/0634; C12N 2500/62; C12N 2500/84

(57) ABSTRACT

The present application relates to the field of cell biology. The present application provides a cryopreservation medium for cryogenic storage of a biological sample, particularly a cryopreservation medium for cryogenic storage of cells in an apheresis sample.

### TECHNICAL FIELD

The present application relates to the field of cell biology. The present application provides a cryopreservation medium for cryogenic storage of a biological sample, particularly a cryopreservation medium for cryogenic storage of cells in an apheresis sample.

### BACKGROUND ART

With an extensive application of cell therapy in clinical practice, the preservation and cryogenic transportation of cell products are facing great challenges.

Cell cryopreservation medium directly affects cell viability after cryopreservation. In the application of clinical tumor treatment, an immune cell preparation enter a patient through venous reinfusion, therefore quality control is a critical link. In clinical application, a single collection of immune cells can be conducted to prepare sufficient number of immune cells for therapy, and the immune cells are then used for multiple reinfusions in batches according to the treatment conditions of a patient. Therefore, an immune cell cryopreservation medium of being safe, efficient and at clinical application level, is one of the important foundations to ensure the therapeutic effect of immune cells. Dimethyl sulfoxide (DMSO) is currently the best cell cryopreservation protectant, while it is also a highly cytotoxic and genotoxic chemical agent. Therefore, it is especially important to find a certain concentration of DMSO that can both perform cryogenically protective functions on cells and minimize its toxicity.

### SUMMARY OF APPLICATION

One object of the present application is to provide a novel cryopreservation medium for a biological sample with better cryopreservation effect and better safety.

In a first aspect of the present application, provided is a cryopreservation medium for a biological sample comprising cryoprotectant and cryopreservation medium base solution, wherein the cryoprotectant comprises any one or a combination of more selected from the group consisting of: DMSO, glycerol and ethylene glycol, and the concentration of the cryoprotectant is about 1.0% to 6.0% (w/v) in the cryopreservation medium for a biological sample.

In a particular embodiment of the above cryopreservation medium, the cryoprotectant is DMSO and has a concentration of about 2,0% to 6,0%, or about 2.0% to 4.5%, or about 2.0% to 4.0%, or about 2.0% to 3.8%, or about 2.0% to 3.75%, or about 2.0% to 3.0%, or about 2.5% to 4.5%, or about 2.5% to 4.0%, or about 2.5% to 3.8%, or about 2.5% to 3.75%, or about 2.5% to 3.0%, or about 3.0% to 4.5%, or about 3.0% to 4.0%, or about 3.0% to 3.8%, or about 3.0% to 3.75%, or about 3.75% to 4.5%, or about 3.8% to 4.5%, or about 3.75% to 4.0%, or about 3.8% to 4.0%, or about 4.0% to 4.5% (w/v) in the cryopreservation medium for a. biological sample.

In a particular embodiment of the above cryopreservation medium, the cryopreservation medium further comprises FBA, wherein the HSA has a concentration of about 1.0% to 6.0%, or about 2.0% to 5.0%, or about 2.0% to 4.0%, or about 2.5% to 5.0%, or about 2.5% to 4.0%, or about 4.0% to 5.0% (w/v) in the cryopreservation medium for a biological sample; preferably, the HAS comprises recombinant human albumin and/or human serum albumin; preferably, the HAS is human serum albumin.

In a particular embodiment of the above cryopreservation medium, in the cryopreservation medium for a biological sample, the DMSO has a concentration of about 2.0%, or about 3.0%, or about 3.8%, or about 3.75%, or about 4.0%, or about 4.5% (w/v), and/or the USA has a concentration of about 2.0%, about 2.5%, about 4.0%, or about 5.0% (w/v).

In a particular embodiment of the above cryopreservation medium, the DMSO has a concentration of about 2.0% (w/v), and the HSA has a concentration of about 2.0% (w/v); or the DMSO has a concentration of about 2.5% (w/v), and the HSA has a concentration of about 2.5% (w/v); or the DMSO has a concentration of about 3.0% (w/v), and the HSA has a concentration of about 2.0% (w/v); or the DMSO has a concentration of about 3.0% (w/v), and the FBA has a concentration of about 4.0% (w/v); or the DMSO has a concentration of about 4.0% (w/v), and the USA has a concentration of about 2.0% (w/v), or the DMSO has a concentration of about 4.5% (w/v), and the HSA has a concentration of about 2.0% (w/v); or the DMSO has a concentration of about 4.0% (w/v), and the HSA has a concentration of about 4.0% (w/v); or the DMSO has a concentration of about 3.8% (w/v), and the HSA has a concentration of about 5.0% (w/v); or

the DMSO has a concentration of about 3.75% (w/v), and the HSA has a concentration of about 5.0% (w/v).

In a particular embodiment of the above cryopreservation medium, the cryopreservation medium base solution is any one, two, or three selected from phosphate buffered saline (PBS), CryoStor® CS5, CryoStor® CS2 and CryoStor®0 CS10, or any combination thereof.

In a particular embodiment of the above cryopreservation medium, the cryopreservation medium for a biological sample is prepared by adjusting the concentration of the cryoprotectant in the cryopreservation medium base solution to an extent that the cryoprotectant has a concentration of about 1.0% to 6.0% (w/v) in the cryopreservation medium for a biological sample.

In a particular embodiment of the above cryopreservation medium, the cryopreservation medium for a biological sample is prepared by adjusting the concentrations of the cryoprotectant and the human serum albumin (HSA) in the cryopreservation medium base solution to an extent that the cryoprotectant has a concentration of about 1.0% to 6.0% (w/v), and the human serum albumin (HSA) has a concentration of about 1.0% to 6.0% in the cryopreservation medium for a biological sample.

In a particular embodiment of the above cryopreservation medium, the cryopreservation medium for a biological sample is prepared by adjusting the concentration of the DMSO in the cryopreservation medium base solution to an extent that the DMSO has a concentration of about 1.0% to 6.0% (w/v) in the cryopreservation medium for a biological sample, for example, adjusting the concentration of DSMO to be about 2.0% to 6.0%, or about 2.0% to 4.5%, or about 2.0% to 4.0%, or about 2.0% to 3.8%, or about 2.0% to 3.0%, or about % to 4.5%, or about 3.0% to 4.0%, or about 2.5% to 3.8%, or about 2.5% to 3.0%, or about 3.0% to 4.5%, or about 3.0% to 4.0%, or about 3.0% to 3.8%, or about 3.8% to 4.5%, or about 3.8% to 4.0%, or about 4.0% to 4.5% (w/v) in the cryopreservation medium for a biological sample; or for example, adjusting the concentration of the DMSO to be about 2.0%, or about 3.0%, or about 3.8%, or about 4.0%, or about 4.5% (w/v) the cryopreservation medium for a biological sample; and/or the HSA has a concentration of about 2.0%, about 2.5%, about 4.0%, or about 5.0% (w/v).

In a particular embodiment of the above cryopreservation medium, the cryopreservation medium for a biological sample is prepared by adjusting the concentrations of the cryoprotectant and the human serum albumin (HSA) in the cryopreservation medium base solution to an extent that the cryoprotectant has a concentration of about 1.0% to 6.0% (w/v), and the human serum albumin (HSA) has a concentration of about 1.0% to 6.0% in the cryopreservation medium for a biological sample. For example, the concentration of human serum albumin (HSA) is adjusted to be about 1.0% to 6.0%, or about 2.0% to 5.0%, or about 2.0% to 4.0%, or about 2.5% to 5.0%, or about 2.5% to 4.0%, or about 4.0% to 5.0% (w/v) in the cryopreservation medium for a biological sample.

In a particular embodiment of the above cryopreservation medium, the CryoStor® CS5, or Solution A or a mixture of the CryoStor® CS5 and Solution A has a concentration of about 40-95%, or about 40-90%, or about 40-80%, or about 40-75%, or about 40-60%, or about 40-50%, or about 50-90%, or about 50-80%, or about 50-75%, or about 50-60%, or about 60-90%, or about 60-80%, or about 60-75%, or about 75-90%, or about 75-80%, or about 80-90% the cryopreservation medium.

In a particular embodiment of the above cryopreservation medium, the CryoStor® CS5, or Solution A, or a mixture of the CryoStor® CS5 and Solution A has a concentration of about 40%, or about 50%, or about 60%, or about 75%, or about 80%, or about 90% (v/v) in the cryopreservation medium.

In a particular embodiment of the above cryopreservation medium, the HSA has a concentration of about 5.0% (w/v); and the CryoStor® CS5, or Solution A, or a mixture of the CryoStor® CS5 and Solution A has a concentration of about 75% (v/v) in the cryopreservation medium; and/or

the HSA has a concentration of about 2.0% (w/v); and the CryoStor® CS5, or Solution A, or a mixture of the CryoStor® CS5 and Solution A has a concentration of about 90% (v/v) in the cryopreservation medium; and/or

the HSA has a concentration of about 4.0% (w/v); and the CryoStor® CS5, or Solution A, or a mixture of the CryoStor® CS5 and Solution A has a concentration of about 80% (v/v) in the preservation medium; and/or

the HSA has a concentration of about 2.5% (w/v); and the CryoStor® CS5, or Solution A, or a mixture of the CryoStor® CS5 and Solution A has a concentration of about 50% (v/v) in the cryopreservation medium; and/or

the HSA has a concentration of about 2.0% (w/v); and the CryoStor® CS5, or Solution A, or a mixture of the CryoStor® CS5 and Solution A has a concentration of about 80% (v/v) in the cryopreservation medium; and/or

the CryoStor® CS5, or Solution A, or a mixture of the CryoStor® CS5 and Solution A has a concentration of about 80% (v/v) in the cryopreservation medium; and/or

the HSA has a concentration of about 4.0% (w/v); and the CryoStor® CS5, or Solution A, or a mixture of the CryoStor® CS5 and Solution A has a concentration of about 60% (v/v) in the cryopreservation medium; and/or

the HSA has a concentration of about 2.0% (w/v); and the CryoStor® CS5, or Solution A, or a mixture of the CryoStor® CS5 and Solution A has a concentration of about 60% (v/v) in the cryopreservation medium; and/or

the CryoStor® CS5, or Solution A, or a mixture of the CryoStor® CS5 and Solution A has a concentration of about 60% (v/v) in the cryopreservation medium; and/or

the HSA has a concentration of about 4.0% (w/v); and the CryoStor® CS5, or Solution A, or a mixture of the CryoStor® CS5 and Solution A has a concentration of about 40% in the cryopreservation medium.

In a particular embodiment of the above cryopreservation medium, the HSA is selected from plasma extracted human albumin, genetically recombinant human albumin, or a combination thereof.

In a particular embodiment of the above cryopreservation medium, the biological sample is or is derived from an apheresis sample, optionally a leukapheresis sample, and/or the sample comprises leukocytes and/or lymphocytes, and/or cells or blood cells in the sample essentially consist of leukocytes, or at least 80%, at least 85%, at least 90%, at least 95%, at least 96%, at least 97%, at least 98%, or at least 99% of the cells in the sample, or at least 80%, at least 85%, at least 90%, at least 95%, at least 96%, at least 97%, at least 98%, or at least 99% of the blood cells in the sample are leukocytes.

In a particular embodiment of the above cryopreservation medium, the biological sample is stored for a period, and after the period the percentage of viable cells in the biological sample is about 24% to about 100%, or at least about 15%, at least about 20%, at least about 25 at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%.

In a particular embodiment of the above cryopreservation medium, the biological sample comprises T cells or engineered T cells; preferably, the biological sample is enriched, comprises CD4⁺T cells or sub-populations thereof and/or CD8⁺T cells or sub-populations thereof, preferably, the T cells are primary T cells; preferably, the cells are derived from an autologous or allogeneic source; preferably, the engineered T cells comprise cells expressing a recombinant molecule or exogenous molecule; preferably, the recombinant molecule or exogenous molecule is optionally a recombinant protein, or optionally a recombinant receptor which is optionally a T cell receptor (TCR), a chimeric receptor, a chimeric antigen receptor (CAR), or a combination thereof.

In a second aspect of the present application, provided is a cell composition, comprising the following components: cells, and the cryopreservation medium according to any one of the embodiments in the first aspect; preferably, the cells are immune cells, mesenchymal stem cells, or a combination thereof; preferably, the cells are peripheral blood mononuclear cell-derived cells.

In a particular embodiment of the above cell composition, the immune cells include, but are not limited to: mononuclear cells, NK cells, B cells, and T cells; preferably, the T cells include, but are not limited to, LAK, TIL, CIK, CTL, CAR-T, and TCR-T;

In a particular embodiment of the above cell composition, preferably, the immune cells comprise T cells or engineered cells; preferably, the immune cells are enriched, and comprises CD4⁺T cells or sub-populations thereof and/or CD8⁺T cells or sub-populations thereof, preferably, the T cells are primary T cells; preferably, the cells are derived from an autologous or allogeneic source; preferably, the engineered T cells comprise cells expressing a recombinant molecule or exogenous molecule; preferably, the recombinant molecule or exogenous molecule is optionally a recombinant protein, or optionally a recombinant receptor which is optionally a T cell receptor (TCR), a chimeric receptor, a chimeric antigen receptor (CAR), or a combination thereof

In a third aspect of the present application, provided is a method for cryopreservation of cells, including the following steps:

(i) mixing cells to he cryopreserved with the cryopreservation medium according to any one of claims 1-12 to obtain a cell composition; and

(ii) cooling the cell composition obtained in the above step (i), then placing in a container at about −80′C to −90° C. or in the gas phase of liquid nitrogen, wherein the container is optionally a bag or a vial.

In a particular embodiment of the above method, the cells to be cryopreserved are cooled under programmed control at a rate of, or about, or greater than 1° C./min, optionally until the temperature reaches about −80° C. to −90° C.

It is understood that, within the scope of the present application, technical features of the present application above and technical features specifically described hereinafter (e.g., in Examples) may be combined with each other to form novel or preferred technical solutions, which are not repeated herein due to limited space.

### BRIEF DESCRIPTION OF THE DRAWINGS

FIG. 1 shows cell viability of cells cryopreserved with cell cryopreservation media CryoStor® CS2, CryoStor® CS5, and CryoStor® CS10 at 0 h after recovery and 8 h after recovery.

FIG. 2 shows expansion folds and cell viability of cells cryopreserved with cell cryopreservation media CryoStor® CS2, CryoStor® CS5, and CryoStor® CS10 for inoculating to culture for 48 h after recovery.

FIG. 3 shows cell viability of cells cryopreserved with Formulas 1, 2, 3, and 4 at 0 h after recovery and 8 h after recovery.

FIG. 4 shows cell viability of cells cryopreserved with Formulas 2, 5, 6, 7, 8, 9, 10, 11, and 12 at 0 h after recovery and 24 h after recovery.

FIG. 5 shows expansion folds and cell viability of cells cryopreserved with Formulas 2, 5, 6, 7, 8, 9, 10, 11, and 12 for culturing for 48h after recovery.

### DETAILED DESCRIPTION OF THE APPLICATION

In view of shortcomings and deficiencies of the prior art, the present application provides a clinical-grade cryopreservation medium with low DMSO for CAR-T cells and a cryopreservation method thereof, wherein the cryopreservation medium of the present application effectively protects cryopreserved cells from freezing injuries while reducing the concentration of DMSO in commercial cryopreservation medium CryoStor® CS5. The cryopreservation medium of the present application has high safety, and shows higher cell viability after recovery than that of the corresponding commercial cryopreservation medium. The cryopreservation method provided in the present application optimizes the cryopreservation-recovery effect for CAR-T cells, and achieves easy operations, thus providing a reliable guarantee for storage and application of clinical CAR-T cells. In one embodiment, CryoStor® CS2 is mixed with CryoStor® CS10 in a volume ratio of 5:3 to prepare a cryopreservation medium base solution having the same composition as CryoStor® CS5, and finally mixed with DMSO and HAS to prepare a cryopreservation medium. In one embodiment, the addition of 2-5% HSA (w/v) to the cryopreservation medium of the present application, while reducing the concentration of DMSO in the corresponding commercial cryopreservation medium CryoStor® CS5, may further protect the cryopreserved cells from freezing injuries effectively. The cryopreservation medium of the present application has high safety, and shows higher cell viability after recovery than that of the corresponding commercial cryopreservation medium.

Unless defined otherwise, all technical terms, symbols and other technical and scientific terminologies or specialized words used herein are intended to have the same meanings as commonly understood by those skilled in the art. In some instances, terms having a meaning of conventional understanding are defined herein for purposes of illustration and/or ease of reference, and the inclusion of such definitions herein should not be construed as implying a significant difference from the conventional understanding in the art.

All publications mentioned in the present application, including patent documents, academic papers, and databases, are incorporated herein by reference in their entirety for the purpose of realizing the application. If a definition set forth herein differs from or otherwise is inconsistent with a definition set forth in a patent, published application, or other publication that is incorporated herein by reference, the definition set forth herein prevails over the definition set forth in the document that is incorporated herein by reference.

As used herein, the singular forms “a”, “an”, and “the” include plural referents unless explicitly stated in the text otherwise. For example, “a” or “an” means “at least a or an” or “one or more”. It is understood that aspects and variations described herein include “consisting of (the aspects and variations)” and/or “essentially consisting of (the aspects and variations)”.

In this disclosure, various aspects of the claimed subject matter are presented in a range format. It is understood that the description in the range format is merely for convenience and brevity and should not be construed as a hard limit on the scope of the claimed subject matter. Accordingly, the description of a range should be considered as having specifically disclosed all the possible subranges as well as individual numerical values within the range. For example, where a range of values is provided, it is understood that each intervening value between the upper and lower limits of that range, and any other stated or intervening value in that stated range, as well as both upper and lower limit values, is encompassed within the claimed subject matter. The upper and lower limits of these smaller ranges may independently be included in the smaller ranges and are also encompassed within the scope of the claimed subject matter, unless the upper or lower limit of the range is specifically excluded. Where a range is set to include one or both of limit values, the claimed subject matter also includes ranges excluding one or both of the limit values. This applies regardless of the breadth of the range.

As used herein, the term “about” refers to a usual error range for values that is readily known to those skilled in the art. When referring to “about” a value or parameter herein, it includes (and describes) embodiments that are directed to the value or parameter itself. For example, a description with respect to “about X” includes the description of “X”. In some embodiments, “about X” includes a range from 50% X to 150% X, or a range from 60% X to 140% X, or a range from 70% X to 130% X, or a range from 80% X to 120% X, or a range from 90% X to 110% X, or a range from 95% X to 105% X, or a range from 97% X to 103% X. For example, “about 4%” includes 4%, or 2% to 6%, or 2.4% to 5.6%, or 2.8% to 5.2%, or 3.2% to 4.8%, or 3.6% to 4.4%, or 3.88% to 4.12%.

The following detailed description and examples illustrate certain embodiments of the disclosure. Those skilled in the art will recognize that, many variations and modifications are encompassed by the scope of the disclosure. Accordingly, the description of certain embodiments should not be considered as limiting.

Cell therapy is a technique whereby cells are administered to a subject for therapeutic purposes. For any given subject, the cells to be administered may be derived from another person or from the subject per se. The latter case may be referred to as autologous cell therapy, i.e., administrating cells collected from a subject back into the subject. Advantages of autologous cell therapy may include a reduced chance of the subject's body rejecting the administered cells, because the donor from which the cells are collected is the subject per se. For cell therapy, how and when the cells are collected from a donor, and how the cells are treated after collection and prior to administration, may affect the efficacy and availability of the therapy, e.g., how quickly the cells may be administered to a subject when needed. For these purposes, methods, systems, compositions, and product are provided for cryogenic storage of cells and cell compositions and/or engineering the same and/or administering the same to a subject. Among other advantages, the embodiments in some aspects have the advantages of enhancing availability and efficacy of cell therapy, and/or other aspects. These methods may also or alternatively provide benefits for other medical or research procedures utilizing cells collected from a donor. In some embodiments, cryopreserved. CAR-T cells may be used for cellular immunotherapy, either by reinfusion to the donor providing T cells for autologous cell therapy, or by infusion into an allogeneic body for therapy; particularly for antitumor treatment in tumor patients. In some embodiments, cryopreserved engineered T cells may be used for cellular immunotherapy, either by reinfusion to the donor providing T cells for autologous cell therapy, or by infusion into an allogeneic body for therapy; particularly for antitumor treatment in tumor patients.

In one embodiment, the biological sample is a blood sample of or derived from the donor. In one embodiment, the biological sample is or comprises a whole blood sample, a buffy coat sample, a peripheral blood mononuclear cell (PBMC) sample, an unfractionated T cell sample, a lymphocyte sample, a leukocyte sample, an apheresis product, or a leukapheresis product. In one embodiment, the biological sample is or is derived from an apheresis sample, optionally a leukapheresis sample and/or the sample comprises leukocytes and/or lymphocytes; and/or the cells or blood cells in the sample essentially consist of leukocytes, or at least 80%, at least 85%. at least 90%, at least 95%, at least 96%, at least 97%, at least 98%, or at least 99% of the cells or blood cells in the sample are leukocytes. In one embodiment, the biological sample is enriched, and comprises CD4⁺T cells or sub-populations thereof, and/or CD8⁺T cells or sub-populations thereof. In one embodiment, the sub-populations of CD4⁺ cells and/or the sub-populations of CD8⁺ cells are optionally selected from the following cells and combinations thereof: memory cells, central memory T (T_{CM}) cells, effector memory (T_{EM}) cells, stem central memory (T_{SCM}) cells, T effector (T_{E}) cells, effector memory RAT (T_{EMRA}) cells, naive T (T_{N}) cells, regulatory T (T_{REG}) cells, and/or helper T cells; or TH1 cells, TFI2 cells, TH3 cells, TH17 cells, TH9 cells, TH22 cells, follicular helper T cells, α/β T , and δ/γ T cells. In one embodiment, the biological sample comprises primary T cells obtained from the subject. In one embodiment, the biological sample comprises one or more sub-populations of T cells or other types of cells, such as whole cell populations, CDT cells, CD8′⁺ cells, and sub-populations thereof, such as sub-populations defined by: function, activation state, maturity, differentiation potential, expansion, recycling, localization and/or persistence ability, antigen specificity, antigen receptor type, presence in a particular organ or compartment, marker or cytokine secretion profile, and/or degree of differentiation. In one embodiment, the biological sample comprises T cells isolated from the PBMC sample by negative selection for markers expressed on non-T cells, e.g., B cells, mononuclear cells, or other leukocytes, such as CD14. In some aspects, a CD4⁺ or CD8⁺ selection step is used to isolate CD4⁺ helper T cells and CD8⁺ cytotoxic T cells. The CD4⁺ and CD8⁺ populations can be further sorted into sub-populations by positive or negative selection for markers expressed, or expressed at a relatively higher degree on one or more naive T cells, memory T cells, and/or effector T cell sub-populations. In one embodiment, the biological sample comprises T cells and/or engineered T cells. In one embodiment, the engineered T cells comprise T cells that express a recombinant molecule or exogenous molecule, which is optionally a recombinant protein, optionally a recombinant receptor which is optionally a T cell receptor (TCR), a chimeric receptor, a chimeric antigen receptor, or a combination thereof.

Apheresis ;generally refers to procedures used to collect blood from a donor or subject. The procedures may include a process for collecting cells from the blood of the donor. Leukapheresis refers to such procedures in which leukocytes are collected from the blood of the donor. In some embodiments, the embodiments and compositions provided involve collecting a blood sample from the donor, e.g., via apheresis; and in some embodiments, the methods and compositions involve administering compositions, such as a cell therapy composition, to the subject. In some embodiments, the donor and the subject are the same individual. In some embodiments, cells from a donor are administered to different subjects.

“Mesenchymal stern cells” refer to multipotent stem cells with high self-renewal capacity and multipotential differentiation, which are derived from the mesoderm in early development. They widely exist in various tissues throughout the body, can he cultured and expanded in vitro, and can differentiate into nerve cells, osteoblasts, muscle cells, fat cells, and the like, under control of specific conditions.

The term “chimeric receptor” refers to a fusion molecule formed by connecting DNA fragments or corresponding cDNA of proteins from different sources through gene recombination technology, wherein the fusion molecule comprises an extracellular domain, a transmembrane domain, and an intracellular domain.

The term “TCR receptor” includes various recombinant proteins derived from TCR, and it comprises an extracellular antigen-binding domain (also referred to as an antigen recognition unit), a TCR transmembrane domain, and an intracellular domain. It is generally capable of i) binding to the surface antigen on a target cell, ii) interacting with other polypeptide components of an intact TCR complex when localized in a T cell. In some embodiments, the TCR-T cell is an NK cell. In some embodiments, the TCR-T cell is a γ δ T cell. In one aspect, the antigen-binding domain of TCR comprises antibody fragments. In another aspect, TCR comprises antibody fragments comprising scFv or sdAb. In one embodiment, the antigen-binding domain of TCR consists of an antibody heavy chain variable region and an antibody light chain variable region respectively fused to the constant regions of α and β chains of the TCR subunit.

The term “chimeric antigen receptor” (CAR) comprises an extracellular antigen-binding domain, a transmembrane domain, and an intracellular signaling domain. The intracellular signaling domain comprises a functional signaling domain of a stimulatory molecule and/or co-stimulatory molecule. In one aspect, the stimulatory molecule is the ζ chain that binds to the T cell receptor complex. In one aspect, the cytoplasmic signaling domain further comprises a functional signaling domain of one or more co-stimulatory molecules, e.g., 4-1BB (i.e., CD137), CD27, and/or CD28.

A “CAR-T cell”, i.e., a chimeric antigen receptor T cell, is formed by transfecting a subject's T cell with a chimeric antigen receptor (CAR) capable of recognizing a certain antigen through gene transduction, so that it can express a chimeric antigen receptor (CAR). In one embodiment, the subject is also the donor of CAR-T cells, In some embodiments, the cryopreserved cells are CAR-T cells prepared from human PBMC-derived T cells.

In some embodiments, the cryopreserved cells are CAR-T cells prepared by collecting human PBMC-derived T cells through apheresis. In a particular embodiment, a preparation method for CAR-T cells can be performed according to a preparation method conventional in the art. For example, a lentiviral vector for expression of CAR is constructed to co-transfect 293T cells with a packaging plasmid to package and prepare a lentivirus, then the lentivirus is used to infect activated T cells; e.g., those CAR-T cells and preparation method thereof disclosed in Chinese Patent Application Publication Nos. CN107058354A, CN107460201A, CN105194661A, CN105315375A, CN105713881A, CN106146666A, CN106519037A, CN106554414A, CN105331585A, CN106397593A, CN106467573A, CN104140974A, CN108884459A, CN107893052A, CN108866003A, CN108853144A, CN109385403A, CN109385400A, CN109468279A, CN109503715A, CN109908176A, CN109880803A, CN110055275A, CN110123837A, CN110438082A, CN110468105A, CN109796532A; International Patent Application Publication Nos. WO2017186121A1, WO2018006882A1, WO2015172339A8, WO2018/018958A1, WO2014180306A1, WO2015197016A1, WP2016008405A1, WO2016086813A1, WO2016150400A1, WO2017032293A1, WP2017080377A1, WO2017186121A1, WO2018045811A1, WO2018108106A1, WO2018/219299, WO2018/210279, WO2019/024933, WO2019/114751, WO2019/114762, WO2019/141270, WO2019/149279, WO2019/170147A1, WO 2019/210863, WO2019/219029, WO2019047932A1, WO2020020210A1, WO2020057666A1, WO2020057641A1, WO2020057668A1, WO2020/063988A1, WO 2020/083406A1, WO2020114518A1, WO2020143631A1, WO2020156554A1, WO2020259707 WO2021/027785, WO2021/052496 A1, WO2021/057906 A1, and WO2018/133877. PBMC means peripheral blood mononuclear cells.

The term “cryogenic storage” or cell cryopreservation technology generally refers to storing a biological sample (e.g., a sample comprising cells/T cells/engineered T cells) at a temperature from −196° C. to −80° C. under certain conditions such that upon or after thawing of the stored biological sample, at least part, or most of the cells/T cells/engineered T cells in the sample remain viable and/or retain at least part of their biological functions. In one aspect, at least a certain percentage of cells in a sample, e.g., about or more than about 20%, about 30%, about 40%, about 50%, about 60%, about 70%, about 80%, about 90%, or about 100% of the cells in the sample remain viable, and/or targeting at markers or indicators of their apoptosis, upon or after thawing of the stored cell sample. Cell cryopreservation medium is the key factor in cell cryopreservation, under slow freezing conditions it enables intracellular water to permeate out of cells before freezing, so as to reduce the formation of ice crystals during the storage at low temperature.

The “cryopreservation medium” or “freezing median” of the present application refers to such a solution when mixed with a sample comprising cells, e.g., an apheresis sample, it helps retain one or more biological functions of the cells during the processes of cooling, cryogenic freezing and/or cryogenic storage of the sample or cells, providing a safe, protective environment for the processes of freezing. storage and thawing of a variety of cells or tissues. In particular, it provides a safe, protective environment for the processes of freezing, storage and thawing of immune cells. As a preferred embodiment, the immune cells are selected from T cells or T cell-based products. Particularly, the T cell-based products are selected from the group consisting of: LAK (lymphokine-activated killer cells), TIL (tumor-infiltrating lymphocytes), CIK (multiple cytokine-induced killer cells), CTL (cytotoxic T lymphocytes), CAR-T (chimeric antigen receptor-modified T cells) and TCR-T (T cell receptor chimeric T cells), etc. Further, the cells are animal cells, more preferably human cells. As a preferred embodiment, the cell cryopreservation medium is used for cryopreservation of CAR-T cells.

The term “cell recovery” refers to the process of reactivation of resting cells. A procedure well known to a person skilled in the art, i.e., the rapid recovery method, is generally used; particularly, the freezing tube/bag is quickly transferred from liquid nitrogen or a temperature of −196° C. to −80° C. into a warm water bath, preferably at 37° C. to 40° C., shaking to accelerate thawing; after the cells are completely thawed, the freezing tube/bag is disinfected; the thawed cryopreservation medium is removed, then the cells are resuspended in a culture solution, and transferred to a cell culture flask to culture in a CO₂ incubator; cell viability and vitality is examined. Alternatively, after completely thawing, the cells may be reinfused to the subject immediately or within 2 hours of resting at room temperature.

In some embodiments, the cell sample may comprise a medium for cryogenic storage or vitrification or a solution comprising a cryogenic protectant. Suitable cryogenic protectants (alternatively referred to as cryoprotectants, cryopreservation protectants) include, but are not limited to: dimethyl sulfoxide (DMSO), glycerol, glycol, propylene glycol, ethylene glycol, propylene glycol, polyethylene glycol (PEG), 1,2-propylene glycol (PROH), or combinations thereof. In some embodiments, the cryogenic storage solution may comprise one or more cell-impermeable cryogenic protectants, including, but not limited to: polyvinylpyrrolidone, hydroxyethyl starch, polysaccharide, monosaccharide, alginate, trehalose, raffinose, dextran, human serum albumin, ficoll, lipoprotein, polyvinylpyrrolidone, hydroxyethyl starch, autologous plasma, or combinations thereof. In some embodiments, the cells are suspended in a freezing medium having cryogenic protectant at a final concentration between about 1% and about 20%, between about 1% and about 9%, or between about 1% and about 5% by mass volume. In certain embodiments, the cryogenic protectant has a final concentration of about 1.0%, about 1.5%, about 2.0%, about 2.0%, about 2.5%, about 3.0%, about 3.1%, about 3.1%, about 3.2%, about 3.3%, about 3.4%, about 3.5%. about 3.6%, about 3.7%, about 3.8%, about 3.9%, 4.0%, about 4.1%, about 4.2%, about 4.3%, about 4.4%, about 4.5%, about 4.6%, about 4,7%, about 4.8%, about 4.9%, about 5.0%, about 5.5%, about 6.0%, about 7.0%, about 8.0%, or about 9.0% by mass/volume ratio in the freezing medium.

DMSO is currently the best cell cryopreservation protectant, while it is also a highly cytotoxic and genotoxic chemical agent. In some embodiments, the cryogenic protectant is DMSO. In a particular embodiment, the cells are suspended in a freezing medium having DMSO at a final concentration between about 1% and about 20%, between about 1% and about 9%, or between about 1% and about 5% by mass/volume ratio. In certain embodiments, the DMSO has a final concentration of about 1.0%, about 1.5%, about 2.0%, about 2.0%, about 2.5%, about 3.0%, about 3.1%, about 3.1%, about 3.2%, about 3.3%, about 3.4%, about 3.5%, about 3.6%, about 3.7%, about 3.8%, about 3.9%, 4.0%, about 4.1%, about 4.2%, about 4.3%, about 4.4%, about 4.5%, about 4.6%, about 4.7%, about 4.8%, about 4.9%, about 5.0%, about 5.5%, about 6.0%, about 7.0%, about 8.0%, or about 9.0% by mass/volume ratio in the freezing medium.

The term “HSA” comprises recombinant human albumin and/or human serum albumin. “Recombinant human albumin” refers to genetically recombinant human albumin. Human serum albumin refers to human albumin extracted from plasma In some embodiments, HSA refers to human albumin extracted from plasma. In a particular embodiment, the cells are suspended in a freezing medium having HSA at a final concentration between about 1% and about 9%, between about 1% and about 6%, or between about 1% and about 5% by mass/volume ratio. In certain embodiments, the HSA has a final concentration of about 1.0%. about 1.5%, about 2.0%, about 2.0%, about 2.5%, about 3.0%, about 3.1%, about 3.1%, about 3.2%, about 3.3%, about 3.4%, about 3.5%, about 3.6%, about 3.7%, about 3.8%, about 3.9%, 4.0%, about 4.1%, about 4.2%, about 4.3%, about 4.4%, about 4.5%, about 4.6%, about 4.7%, about 4.8%, about 4.9%, about 5.0%, about 5.5%, about 6.0%, about 7.0%, about 8.0%, or about 9.0% by mass/volume ratio in the freezing medium. In one embodiment, the HSA is a commercial HSA. In one embodiment, the HSA is human albumin from Chengdu Rongsheng Pharmaceutical Co. Ltd.

The term “cryopreseniation medium base solution” refers to cell freezing media other than DMSO and HSA. In one embodiment, the cryopreservation medium base solution is any one, two, or three selected from phosphate buffered saline (PBS), CryoStor® CS5, CryoStor® CS2, and CryoStor® CS10, or any combination thereof. In one embodiment, the cryopreservation medium base solution includes other suitable cell freezing media.

The commercial cell cryopreservation media CryoStor® CS2 (or referred to as CS2), CryoStor® CS5 (or referred to as CS5), and CryoStor® CS10 (or referred to as CS10) have DMSO at a concentration of 2%, 5% and 10% by mass/volume ratio respectively, with other components being the same. In the present application, CryoStor® CS2 (or referred to as CS2). CryoStor® CS5 (or referred to as CS5), and CryoStor® CS10 (or referred to as CS10) are used as the cryopreservation medium base solution to prepare the final cryopreservation media.

By comparison, the viability of cells cryopreserved with CryoStor® CS5 and CryoStor® CS10 after recovery is higher than 90%, while that of cells cryopreserved with CyoStor® CS2 is lower than 90%. Resting at room temperature for 8 hours after recovery, the cell viability of CS5 group is significantly higher than that of CS2 group and CS10 group. This suggests that among the three cryopreservation media, CS5 with DMSO at a concentration of 5% can not only allow DMSO to perform cryogenically protective functions on cells, but also reduce the toxicity of high concentration of DMSO to cells. In cell therapy, due to the limited number or viability of immune cells collected from donors, how to maximize the preservation of cell viability or functions is an important part in cell therapy. In particular, cell therapy for solid tumors often requires the infusion of large doses of immune cells into subjects. The present application aims to further improve the cryogenically protective effects of existing ctyopreservation media on cells and reduce their toxicity to cells. After recovery of cells cryopreserved with cryopreservation medium CS5, their viability and expansion ability in vitro can be significantly improved by adding low dose of HSA and reducing the concentration of DMSO. The CSS cryopreservation medium base solution used in the present application may also be prepared by mixing CS2 and CS10 at a volume ratio of or about 5:3. For example, the cryopreservation medium of the present application with CS5 at a concentration of or about 70% (v/v) may also be prepared by adding CS2 with a concentration of or about 44% (v/v) and CS10 with a concentration of or about 26% (v/v); the cryopreservation medium of the present application with CS5 at a concentration of or about 75% (v/v) may also be prepared by adding CS2 with a concentration of or about 47% (v/v) and CS10 with a concentration of or about 28% (v/v); the cryopreservation medium of the present application with CS5 at a concentration of or about 80% (v/v) may also be prepared by adding CS2 with a concentration of or about 50% (v/v) and CS10 with a concentration of or about 30% (v/v); and the cryopreservation medium of the present application with CS5 at a concentration of or about 90% (v/v) may also be prepared by adding CS2 with a concentration of or about 56% (v/v) and CS10 with a concentration of or about 34% (v/v). In some embodiments, the CryoStor® CS5 or Solution A or a mixture of the CryoStor® CS5 and Solution A has a concentration of or about 40-95%. of or about 40-90%, of or about 40-80%, of or about 40-75%, of or about 40-60%, of or about 40-50%, of or about 50-90% of or about 50-80%, of or about 50-75%, of or about 50-60%, of or about 60-90%, of or about 60-80%, of or about 60-75%, of or about 75-90%, of or about 75-80%, of or about 80-90% (v/v) in the cryopreservation medium. In some embodiments, the CryoStor® CS5 or Solution A or a mixture of the CryoStor® CS5 and Solution A has a concentration of or about 40%, of or about 50%, of or about 60%, of or about 75%, of or about 80%, of or about 90% in the cryopreservation medium. In some embodiments, the HSA has a concentration of or about 5.0% (w/v), and the CryoStor® CS5 or Solution A or a mixture of the CrvoStor® CS5 and Solution A has a concentration of or about 75% (v/v) in the cryopreservation medium; and/or the HSA has a concentration of or about 2.0% (w/v), and the CryoStor® CS5 or Solution A or a mixture of the CryoStor® CS5 and Solution A has a concentration of or about 90% (v/v) in the cryopreservation medium; and/or the HSA has a concentration of or about 4.0% (w/v), and the CryoStor® CS5 or Solution A or a mixture of the CryoStor® CS5 and Solution A has a concentration of or about 80% (v/v) in the ctyopreservation medium; and/or the HSA has a concentration of or about 2.5% (w/v), and the CryoStor® CS5 or Solution A or a mixture of the CryoStor® CS5 and. Solution A has a concentration of or about 50% (v/v) in the cryopreservation medium; and/or the HSA has a concentration of or about 2.0% (w/v), and the CryoStor® CS5 or Solution A or a mixture of the CryoStor® CS5 and Solution A has a concentration of or about 80% (v/v) in the cryopreservation medium; and/or the CryoStor® CS5 or Solution A or a mixture of the CryoStor® CS5 and Solution A has a concentration of or about 80% (v/v) in the cryopreservation medium; and/or the HSA has a concentration of or about 40% (w/v), and the CryoStor® CS5 or Solution A or a mixture of the CryoStor® CS5 and Solution A has a concentration of or about 60% (v/v) in the cryopreservation medium; and/or the HSA has a concentration of or about 2.0% (w/v), and the CryoStor® CS5 or Solution A or a mixture of the CryoStor® CS5 and Solution A has a concentration of or about 60% (v/v) in the cryopreservation medium; and/or the CryoStor® CS5 or Solution A or a mixture of the CryoStor® CS5 and Solution A has a concentration of or about 60% in the cryopreservation medium; and/or the HSA has a concentration of or about 4.0% (w/v and the CryoStor® CS5 or Solution A or a mixture of the CryoStor® CS5 and Solution A has a concentration of or about 40% (v/v) in the cryopreservation medium.

Methods for preparation of the cryopreservation medium base solution by using CryoStor® CS2 (or referred to as CS2), CryoStor® CS5 (or referred to as CS5), and CryoStor®0 CS10 (or referred to as CS10) are recited above. However, in practice, one, two or three selected from CryoStor® CS5, CryoStor® CS2, and CryoStor® CS1 0, or any combination thereof may be used for preparation the cryopreservation medium base solution. Meanwhile, when only CryoStor® CS2 is used, DMSO may be added to change the concentration of DMSO, in this case the effects of the cryopreservation medium may generally not be affected even if the other components in the base solution are slightly diluted. The core concept for preparation of the cryopreservation medium according to the present application is using CryoStor® CS5, CryoStor® CS2 and CryoStor® CS10 as the cryopreservation medium base solution, while controlling the concentration of DMSO and/or HAS in the prepared cryopreservation medium.

In certain embodiments, the cells are suspended in the freezing medium at a density: between about 1×10⁵ cells/mL and about 2×10⁸ cells/mL, between about 1×10⁵ cells/mL and about 1×10⁸ cells/mL, between about 1×10⁶ cells/mL and about 1×10⁸ cells/mL, between about 1×10⁷ cells/mL and about 1×10⁸ cells/mL, or between about 4×10⁷ cells/mL and 1×10⁸ cells/mL. In certain embodiments the cells are suspended in the freezing medium at a density of: about 1×10⁵ cells/mL, about 5×10⁵ cells/mL, about 1×10⁶ cells/mL, about 2×10⁶ cells/mL, about 5×10⁶ cells/mL, about 1×10⁷ cells/mL, about 1.5×10⁷ cells/mL, about 2×10⁷ cells/mL, about 2.5×10⁷ cells/mL, about 3×10⁷ cells/mL, about 3.5×10⁷ cells/mL, about 4×10⁷ cells/mL, about 4.5×10⁷ cells/mL, about 5.0×10⁷ cells/mL, about 5.5×10⁷ cells/mL, about 6.0×10⁷ cells/mL, about 6.5×10⁷ cells/mL, about 7.0×10⁷ cells/mL, about 7.5×10⁷ cells/mL, about 8.0×10⁷ cells/mL, about 8.5×10⁷ cells/mL, about 9.0×10⁷ cells/mL, about 9.5×10⁷ cells/mL, about 1/1 O^{s} cells/mL, about 1.5×10⁸ cells/mL, or about 2×10⁸ cells/mL. In certain embodiments, the cells are suspended in the freezing medium at a density between about 1.5×10⁷ cells/mL and about 6×10⁷ cells/mL. in certain embodiments, the cells are suspended in the freezing medium at a density between about 5×10⁶ cells/mL and about 150×10⁶ cells/mL. In certain embodiments, the cells are suspended in the freezing medium at a density of at least about 1×10⁷ cells/mL. In a particular embodiment, the cells are suspended in the freezing medium at a density of at least about 5.0×10⁷ cells/mL. In some embodiments, the cells are viable cells. In some embodiments, the cells are viable CAR-T cells, in some embodiments, the cell density is determined by T cell diameter.

In some embodiments, the cells are stored or preserved for a period greater than or equal to 12 hours, 24 hours, 36 hours, or 48 hours. In some embodiments, the cells are stored or preserved for a period greater than or equal to 1 week, 2 weeks, 3 weeks, or 4 weeks. In some embodiments, the cells are placed in long-term storage or long-term preservation. In some aspects, the cells are stored for a period greater than or equal to 1 month, 2 months, 3 months, 4 months, 5 months, 6 months, 7 months, 1 months, 9 months, 10 months, 11 months, 1 year, 2 years, 3 years, 4 years, 5 years, 6 years, 7 years, 1 years, 9 years, 10 years, 11 years, 12 years, 13 years, 14 years, 15 years, 16 years, 17 years, 18 years, 19 years, 20 years, 25 years, 30 years, 35 years, 40 years, or longer.

In some embodiments, the subject or donor is a mammal, such as a human or other animal, typically a human. In some embodiments, the subject (e.g., the patient) to which the cells or the cell composition are administered, is a mammal, typically a primate, such as a human. In some embodiments, the primate is a monkey or ape. The subject may be male or female, and may be of any suitable age, including infant, juvenile, adolescent, adult and/or elderly subjects. In some embodiments, the subject is a non-primate mammal, such as a rodent.

In some embodiments, the cell composition is encapsulated in one or more bags suitable for cryogenic storage (e.g,, freezing bags from Miltenyi Biotec). In some embodiments, the cell composition is encapsulated in one or more vials or tubes suitable tor cryogenic storage (e.g., tubes from Thermo Fisher).

In some embodiments, the methods provided include an incubation, culture, and/or genetic engineering step before or after the cryogenic storage step. In some embodiments, at least the genetic engineering step is carried out after the cryogenic storage step. For example, in some embodiments, methods are provided for incubating and/or engineering the cryogenically stored cell populations.

In some embodiments, the cells are frozen, for example, at a particular cell density, such as a known or controlled cell density. In certain embodiments, the cell density during the freezing process may affect cell death and/or cell damage that occurs during and/or as a result of the freezing process.

For example, in a particular embodiment, the cell density affects equilibrium, such as osmotic equilibrium with the environment during the freezing process. In some embodiments, the equilibrium is, includes, and/or results in dehydration. In certain embodiments, the dehydration is or includes cell dehydration, which occurs upon contacting, mixing and/or incubating with a freezing medium, such as DMSO and/or DMSO-containing solutions. In a particular embodiment, the dehydration is or includes dehydration caused by nucleation and growth of ice crystals in the extracellular space, such as by reduction of the effective liquid water concentration exposed to the cells. In some embodiments, the cells are frozen at a cell density resulting in a dehydration that is slower and/or at a lower speed than cells frozen at a different (e.g., higher or lower) cell density. In some embodiments, compared with the cells frozen at a different (e.g., higher or lower) cell density under the same or similar conditions, the cells are frozen at a cell density resulting in a dehydration that is about 5%, about 10%, about 20%, about 25%, about 30%, about 40%, about 50%, about 60%, about 70%, about 80%, about 90%, about 100%, about 125%, about 150%, about 175%, about 200%, about 1-fold, about 2-fold, about 3-fold, about 4-fold, about 5-fold, about 10-fold, about 50-fold, or about 100-fold slower; or is at least 5%, at least 10%, at least 20%, at least 25%, at least 30%, at least 40%, at least 50%, at least 60%. at least 70%, at least 80%, at least 90%, at least 100%, at least 125%, at least 150%, at least 175%, at least 200%. at least 1-fold, at least 2-fold, at least 3-fold, at least 4-fold, at least 5-fold, at least 10-fold, at least 50-fold, or at least 100-fold slower; or 5%, 10%, 20%, 25%, 30%, 40%, 50%. 60%, 70%, 80%, 90%, 100%, 125%, 150%, 175%, 200%, 1-fold, 2-fold, 3-fold, 4-fold, 5-fold, 10-fold, 50-fold, or 100-fold slower.

In some embodiments, the cells are frozen in one or more containers. In certain embodiments, the containers are freezing containers and/or cryogenically protective containers. Containers suitable for cryogenic freezing include, but are not limited to: vials or tubes, bags, such as plastic bags, and canes. In a particular embodiment, the cells, e.g., the same cell composition (such as a cell composition comprising cells expressing CAR) are frozen in 1, 2, 3, 4, 5, 6, 7, 8, 9, 10, or more than 10 individual containers. For example, in some embodiments, the cells and/or the cell composition are suspended in a volume of, such as, for example, solution, freezing medium, and/or cryogenic protectant, wherein the volume is greater than a volume appropriate for the container, and thus the volume is placed in two or more containers. In some embodiments, the volume is 100 mL, 50 mL, 25 mL, 20 mL, 15 nit, 10 mL, 5 mL, or less than 5 mL, or is about 100 mL, about 50 mL, about 25 mL, about 20 mL, about 15 mL, about 10 mL, about 5 mL, or about less than 5 mL; or is less than 100 mL, less than 50 mL, less than 25 mL, less than 20 mL, less than 15 mL, less than 10 mL, less than 5 mL, or less than 5 mL, and the cells are frozen in two, three, four, five, six, seven, eight, nine, ten, or more than ten individual vials or tubes. In a particular embodiment, the cells of the same volume are placed into each of the vials or tubes. In some embodiments, the vials or tubes are the same vials or tubes, e.g., vials or tubes of the same production, model, and/or manufacturing batch. In a particular embodiment, the volume is 10 mL, 15 mL, 20 mL, 25 mL, 30 mL, 40 mL, 50 mL, 60 mL, 70 mL, 80 mL, 90 mL, 100 mL, 120 mL, 150 mL, 200 mL, or more than 200 mL; or is about 10 mL, about 15 mL, about 20 mL, about 25 mL, about 30 mL, about 40 mL, about 50 mL, about 60 mL, about 70 mL, about 80 mL, about 90 mL, about 100 mL, about 120 mL, about 150 mL, about 200 mL, or about more than 200 mL; or is greater than 10 mL, greater than 15 mL, greater than 20 mL, greater than 25 mL, greater than 30 mL, greater than 40 mL, greater than 50 mL, greater than 60 mL, greater than 70 mL, greater than 80 mL, greater than 90 mL, greater than 100 mL, greater than 120 mL, greater than 150 mL, greater than 200 mL, or greater than 200 mL; and the cells are frozen in two, three, four, five, six, seven, eight, nine, ten, or more than ten individual bags. In a particular embodiment, the cells of the same volume are placed into each of the bags. In some embodiments, the bags are the same bags, e.g., bags of the same production, model, and/or manufacturing batch.

In some embodiments, a container is a vial or tube. In certain embodiments, the container is a vial or tube with a filling volume of 0.5 mL, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 25 mL, 30 mL, 35 mL, 40 mL, 45 mL, or 50 mL; or a vial or tube with a filling volume of about 0.5 mL, about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 25 mL, about 30 mL, about 35 mL, about 40 mL, about 45 mL or about 50 mL; or a vial or tube with a tilling volume of at least 0.5 mL, at least 1 mL, at least 2 mL, at least 3 mL, at least 4 mL, at least 5 mL, at least 6 mL, at least 7 mL, at least 8 mL, at least 9 mL, at least 10 mL, at least 11 mL, at least 12 mi, at least 13 mL, at least 14 mL, at least 15 mL, at least 16 mL, at least 17 nit, at least 18 nit, at least 19 mL, at least 20 mL, at least 25 mL, at least 30 mL, at least 35 mL, at least 40 mL, at least 45 mL, or at least 50 mL. In some embodiments, the vial or tube has a filling volume between 1 mL and 120 mL, between 1 mL and 20 mL, between 1 mL and 5 mL, between 1 mL and 10 mL, between 1 mL and 40 mL, or between 20 mL, and 40 mL, and each of the above ranges includes the endpoints thereof. In some embodiments, the vial or tube is a freezing vial or tube, a cryogenically protective vial or tube, and/or a cryovial.

In a particular embodiment, a container is a bag. In certain embodiments, the container is a bag with a filling volume of 0.5 mL, 1 mL, 2 mL, 3 mL, 4 mL, 5 rid 6 mL, 7 mL, 9 mL, 10 mL, 11 mL, 12 mL, 13 mL, 14 mL, 15 mL, 16 mL, 17 mL, 18 mL, 19 mL, 20 mL, 25 mL, 30 mL, 35 mL, 40 mL, 45 mL, or 50 mL; or a bag with a filling volume of about 0.5 mL, about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 25 mL, about 30 mL, about 35 mL, about 40 mL, about 45 mL, or about 50 mL; or a bag with a filling volume of at least 0.5 mL, at least 1 mL, at least 2 mL, at least 3 mL, at least 4 mL, at least 5 mL, at least 6 mL, at least 7 mL, at least 8 mL, at least 9 mL, at least 10 mL, at least 11 mL, at least 12 mL, at least 13 mL, at least 14 mL, at least 15 mL, at least 16 mL, at least 17 mL, at least 18 mL, at least 19 mL, at least 20 mL, at least 25 mL, at least 30 mL, at least 35 mL, at least 40 at least 45 mL, or at least 50 mL. In some embodiments, the bag has a filling volume between 1 mL and 120 mL, between 1 mL and 20 mL, between 1 mL and 5 mL, between 1 mL and 40 mL, between 20 mL and 40 mL, between 1 mL and 70 mL, or between 50 mL and 70 mL, and each of the above ranges includes the endpoints thereof. In some embodiments, the bag is filled with a volume of 100 mL, 75 mL, 70 mL, 50 mL, 25 mL, 20 mL, or 10 mL, or a volume of about 100 mL, about 75 mL, about 70 mL, about 50 mL, about 25 mL, about 20 mL, or about 10 mL; or a volume of less than 100 mL, less than 75 mL, less than 70 mL, less than 50 mL, less than 25 mL, less than 20 mL, or less than 10 mL. Suitable bags are known and include, but are not limited to, freezing bags from Miltenyi Biotec. In certain embodiments, the volume is a volume at room temperature. In some embodiments, the volume is a volume between 4° C. and 37° C., between 16° C. and 27° C. (including the endpoints); or a volume at 16° C., 17° C., 13° C., 19° C., 20° C., 21° C., 22° C., 23′C, 24° C., 25° C., 26° C., 27° C., 28° C., 29° C., 30° C., 31° C., 32° C., 33° C., 34° C., 35° C., 36° C., or 37° C.: or a volume at about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C.; or a volume of at least 16° C., at least 17° C. at least 18° C., at least 19° C., at least 20° C., at least 21° C., at least 22° C., at least 23° C. at least 24° C. at least 25° C., at least 26° C., at least 27° C., at least 28° C., at least 29° C., at least 30° C., at least 31° C., at least 32° C., at least 33° C., at least 34° C., at least 35° C., at least 36° C., or at least 37° C. In some embodiments, the volume is a volume at 25° C.

In some embodiments, cells in a medium or solution (e.g., a freezing solution) of a volume between 1 mL and 20 mL (including the endpoints) are frozen in one or more vials or tubes. In some embodiments, the one or more vials or tubes have a filling volume between 1 mL and 5 mL (including the endpoints). In certain embodiments, cells in a medium or solution(e.g., a freezing medium) of a volume between 20 mL and 120 mL (including the endpoints) are frozen in one or more bags. In a particular embodiment, the one or more bags have a filling volume between 20 mL and 40 mL (including the endpoints). In some embodiments, cells in a medium or solution (e.g., a freezing medium) of a volume of 120 mL or greater are frozen in one or more bags. In certain embodiments, the one or more bags have a filling volume between 50 mL and 70 mL (including the endpoints).

In certain embodiments, the cells are frozen in a solution (e.g., a freezing medium) which is placed in a container (e.g., a bag or vial or tube) having a certain ratio of surface area to volume. In a particular embodiment, the ratio of surface area to volume is from 0.1 cm⁻¹ to 100 cm⁻¹, 1 cm⁻¹ to 50 cm⁻¹, 1 cm⁻¹ to 20 cm⁻¹, 1 cm⁻¹ to 10 cm⁻¹, 2 cm⁻¹ to 10 cm⁻¹, 3 cm⁻to 7 cm⁻¹, or 3 cm⁻¹ to 6 cm⁻¹, or from about 0.1 cm⁻¹ to about 100 cm⁻¹, about 1 cm⁻¹ to about 50 cm⁻¹, about 1 cm⁻¹ to about 20 cm⁻¹, about 1 cm⁻¹ to about 10 cm⁻¹, about 2 cm⁻¹ to about 10 cm⁻¹, about 3 cm⁻¹ to about 7 cm⁻¹, or about 3 cm⁻¹ to about 6 cm⁻¹, and each of the above ranges includes the endpoints thereof. In a particular embodiment, the ratio of surface area to volume is between 3 cm⁻¹ and 6 cm⁻¹, or between about 3 cm⁻¹ and about 6 cm⁻¹. In some embodiments, the ratio of surface area to volume is 3 cm⁻¹, 4 cm⁻¹, 5 cm⁻¹, 6 cm⁻¹, or 7 cm⁻¹; or is about 3 cm⁻¹, about 4 cm⁻¹, about 5 cm⁻¹, about 6 cm⁻¹, or about 7 cm⁻¹; or is at least 3 cm⁻¹, at least 4 cm⁻¹, at least 5 cm⁻¹, at least 6 cm⁻¹, or at least 7 cm⁻¹.

In some embodiments, the cells are frozen to −80° C. at a rate of, or about, or greater than 1° C./min. In some embodiments, the cells are actively and/or efficiently cooled at a rate of, or about, or greater than 1° C./min with a controlled-rate freezer. In some embodiments, the cells may be frozen with the controlled-rate freezer. In sonic aspects, the controlled-rate freezer is used to freeze cells with a programmed cooling profile, e.g., a cooling profile having multiple cooling and/or heating rates. Such a cooling profile may be programmed to control nucleation, e.g., ice formation, so as to for example reduce intracellular ice formation. In some embodiments, the temperature selected to begin the rapid cooling profile and the temperature to finish the cooling are related to the type of container and the volume for freezing. In some embodiments, if the volume is excessively small or the container has an excessively high ratio of surface area to volume, the sample may respond excessively fast to a temperature decrease, be frozen excessively fast, and be at risk of intracellular ice formation. In other embodiments, if the volume is excessively large or the container has an excessively low ratio of surface area to volume, the sample may not respond to a temperature decrease, be frozen excessively slowly, and be at risk of uncontrolled nucleation in the late cooling profile, as well as damage of solution effect caused by prolonged exposure to cryogenic protectants (e.g., DMSO) prior to ice crystal formation.

In some embodiments, cells are frozen with the cooling profile as follows: a holding step at 4.0° C., followed by a cooling step at a rate of 1.2° C./min, until the sample reaches a temperature of −6° C. In some aspects, the sample is then cooled at a rate of 25′C./min. until the container containing the sample reaches −65° C. In some aspects, the sample is then heated at a rate of 5° C./min until the container containing the sample reaches −30° C. In some aspects, the sample is then cooled at a rate of PC/min until the container containing the sample reaches −40° C. in sonic aspects, the sample is then cooled at a rate of 1° C./min until the container containing the sample reaches −90° C. In sonic aspects, the sample is then maintained at −90° C. until being removed from the controlled-rate freezer.

In some embodiments, cells are frozen with the cooling profile as follows: a holding step at 4.0° C., followed by a cooling step at a rate of 1.2° C./min, until the sample reaches a temperature of −6° C. In some aspects, the sample is then cooled at a rate of 25° C./min until the container containing the sample reaches −65° C. In some aspects, the sample is then heated at a rate of 15′C,/min until the container containing the sample reaches −30° C. In some aspects, the sample is then cooled at a rate of 1° C./min until the container containing the sample reaches −40° C. In some aspects, the sample is then cooled at a rate of 10° C./min until the container containing the sample reaches −90° C. In some aspects, the sample is then maintained at −90° C. until being removed from the controlled-rate freezer.

In some embodiments, the cells are cooled to a temperature from above −80° C. to 0° C. prior to cryogenic freezing and/or storage. For example, the cells may be cooled to −20° C., or to a temperature above −80° C. or below −20° C.

In some embodiments, the cells are cryogenically frozen to a temperature from −210° C. to −80° C. prior to cryogenic storage. For example, the cells may be cryogenically frozen to −210′C, or −196° C., or −80° C.

In some embodiments, the cells are cooled and/or cryogenically frozen at a rate of 0.1° C./min to 5° C./min. In some embodiments, the cells are cooled and/or cryogenically frozen at a rate of 0.2° C./min to 4° C./min. In some embodiments, the cells are cooled and/or cryogenically frozen at a rate of 0.5° C./min to 3° C./min. In some embodiments, the cells are cooled and/or cryogenically frozen at a rate of 0.5° C./min to 2° C./min. In some embodiments, the cells are cooled and/or cryogenically frozen at a rate of 1° C./min. For example, a way of cooling and/or cryogenically freezing the cells at the rates described above includes placing the cells in a programmable refrigerator that reduces the temperature therein at such rates. Another way to accomplish this includes placing the vial with the cells in a container where the vial is surrounded by isopropanol, and placing the container in a cooled or cryogenically frozen environment. In some embodiments, the cells are stored at a temperature lower than the temperature at which the cells are frozen with a step-by-step method. For example, in some embodiments, the cells are stored at a. temperature below −80° C., such as below −100° C., below −110° C., below −120° C., below −130° C., below −340° C., below −150° C., below −160° C., or lower. In some aspects, such storage provides retention of cells or biological activities thereof to a greater extent and/or for a longer period.

In some embodiments, prior to cooling or cryogenic freezing, the cells are washed to remove certain components other than the cells to be stored. For example, the cells are washed to remove plasma and/or platelets. The cells are washed, for example as described in PCT Application Publication WO 2015/164675, which is incorporated herein by reference in its entirety.

In some embodiments, the cells are mixed with a freezing medium prior to cooling, cryogenic freezing, and/or cryogenic storage. In some embodiments, the freezing solution brings a greater retention of one or more biological functions of the cells after cooling, cryogenic freezing or cryogenic storage and after cell thawing, as compared to the cells cooled, cryogenically frozen, or cryogenically stored without the freezing medium.

In some embodiments, the freezing medium comprises DMSO from 0.1% to 50% by mass/volume ratio and HSA from 0.1% to 20% by mass/volume ratio. In some embodiments, the freezing medium comprises DMSO from 0.5% to 40% by mass/volume ratio and HSA from 0.2% to 15% by mass/volume ratio. In some embodiments, the freezing medium comprises DMSO from to 30% by mass/volume ratio and HSA from 0.5% to 10% by mass/volume ratio. In some embodiments, the freezing medium comprises DMSO from 1% to 20% by mass/volume ratio and HSA from 2% to 7% by mass/volume ratio. In some embodiments, the freezing medium comprises DMSO from 1% to 5% by mass/volume ratio and HSA from 1% to 5% by mass/volume ratio. In some embodiments, the freezing medium comprises DMSO of 4% by mass/ volume ratio, or 3.8%, or 4.5%, or 3%, or by mass/volume ratio; and/or RSA of 4% or 2% by mass/volume ratio. In some embodiments, the concentrations above are the concentrations of DMSO and HSA before mixing the freezing medium with the cells. In some embodiments, the concentrations above are the concentrations of DMSO and HSA after mixing the freezing medium with the cells.

In some embodiments, the cells are cryogenically stored at a temperature from −210° C. to −80° C. In some embodiments, the cells are cryogenically stored at a temperature, from −210° C. to −196° C. In some embodiments, the cells are cryogenically stored at a temperature from −196° C. to −80° C. In some embodiments, the cells are cryogenically stored in the gas phase of a liquid nitrogen storage tank.

In some embodiments, the cells are cryogenically stored for a period from 1 day to 12 years. For example, the cells may be stored for a period before they lose vitality for cell therapy, and until they are needed for treatment of a subject. By storing the cells according to the storage methods described herein until they are needed for treatment of a subject, in certain embodiments, the methods disclosed herein provide advantages that the cells are readily available when the subject requires the cells for cell therapy. In some embodiments, the cells are stored or preserved for a period greater than or equal to 12 hours, 24 hours. 36 hours, or 48 hours. In some embodiments, the cells are stored or preserved for a period greater than or equal to I week, 2 weeks, 3 weeks, or 4 weeks. In some embodiments, the cells are placed in a state of “long term storage” or “long term preservation”. In some aspects, the cells are stored for a period greater than or equal to 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years. 40 years, or longer.

In some embodiments, the cells are thawed after the storage period. in some embodiments, the cells are thawed by increasing the temperature of the cells to or above 0° C., in order to restore at least part of the biological functions of the cells. In some embodiments, the cells are thawed by increasing the temperature of the cells to 37° C., in order to restore at least part of the biological functions of the cells. According to certain embodiments, the thawing includes placing the cells in a container in a water bath of 37° C. or 38° C. for 60-90 seconds.

In some embodiments, the cells are thawed. In a particular embodiment, the cells are thawed rapidly, e.g., as rapidly as possible without overheating the cells or exposing the cells to high temperatures such as above 37° C. In some embodiments, rapid thawing reduces and/or prevents exposure of cells to high concentrations of cryogenic protectants and/or DMSO. In a particular embodiment, the rate of thawing process may be affected by the characteristics of the container (e.g., vial and/or bag) in which the cells are frozen and thawed.

In a particular embodiment, the cell are thawed at a temperature of 37° C., 35° C., 32° C., 30° C., 29° C., 28° C., 27° C., 26° C., 25° C., 24° C., 23° C., 22° C., 21° C., 20° C., or 15° C., or between 15° C. and 30° C., between 23° C. and 28° C., or between 24° C. and 26° C.; or at about 37° C., about 35° C., about 32° C., about 30° C., about 29° C., about 28° C., about 27° C., about 26° C., about 25° C., about 24° C., about 23° C., about 22° C., about 21° C., about 20° C., or about 15° C., or between about 15° C. and about 30° C., between about 23° C. and about 28° C., or between about 24° C. and about 26° C.; or less than 37° C., less than 35° C., less than 32° C., less than 30° C., less than 29° C., less than 28° C., less than 27° C., less than 26° C., less than 25° C., less than 24° C., less than 23° C., less than 22° C., less than 21° C., less than 20° C., or less than 15° C., or less than 15-30° C., less than 23-28° C., or less than 24-26° C., and each of the above ranges includes the endpoints thereof.

In some embodiments, the cells are thawed in a dry thawer or in a water bath on a heat block. In certain embodiments, the cells are not thawed in a dry thawer or in a water bath on a heat block. In some embodiments, the cells are thawed at room temperature.

In some embodiments, the thickness of the container wall affects the rate at which the cells thaw, such as, for example, cells in containers with thick walls may thaw at a slower rate than those in containers with thinner walls. In some embodiments, containers with low ratios of surface area to volume may have slow and/or non-uniform thawing rates. In some embodiments, the cryogenically frozen cells are thawed rapidly in a container having a ratio of surface area to volume of: 1 cm⁻¹, 2 cm⁻¹, 3 cm⁻¹, 4 cm⁻, 5 cm⁻¹, 6 cm⁻¹, or 7 cm⁻¹, 8 cm⁻¹, 9 cm⁻¹, or 10 cm⁻¹; or about 1 cm⁻¹, about 2 cm⁻¹, about 3 cm⁻¹, about 4 cm⁻¹, about 5 cm⁻¹, about 6 cm⁻¹, or about 7 cm⁻¹, about 8 cm⁻¹, about 9 cm⁻¹, or about 10 cm⁻¹, or at least 1 cm⁻¹, at least 2 cm⁻¹, at least 3 cm⁻¹, at least 4 cm⁻¹, at least 5 cm⁻¹, at least 6 cm⁻¹, or at least 7 cm⁻¹, at least 8 cm⁻¹, at least 9 cm⁻¹, or at least 10 cm⁻¹. In a particular embodiment, the cells are thawed within 120 minutes, 90 minutes, 60 minutes, 45 minute, 30 minutes, 25 minutes, 20 minutes, 15 minutes, or 10 minutes; or within about 120 minutes, about 90 minutes, about 60 minutes, about 45 minutes, about 30 minutes, about 25 minutes, about 20 minutes, about 15 minutes, or about 10 minutes, or within less than 120 minutes, less than 90 minutes, less than 60 minutes, less than 45 minutes, less than 30 minutes, less than 25 minutes, less than 20 minutes, less than 15 minutes, or less than 10 minutes. In some embodiments, the cells are thawed between 10 minutes and 60 minutes, between 15 minutes and 45 minutes, or between 15 minutes and 25 minutes, and each of the above ranges includes the endpoints thereof. In a particular embodiment, the cells are thawed in 20 minutes, in about 20 minutes, or in less than 20 minutes,

In certain embodiments, the thawed cells are allowed to stand, e.g., to be incubated or cultured, prior to administration or prior to any subsequent engineering and/or treatment steps. In some embodiments, the cells are allowed to stand in the absence of low amount and/or undetectable amount of a cryogenic protectant, or in the presence of a. cryogenic protectant (e.g., DMSO). In a particular embodiment, the thawed cells are allowed to stand after or immediately after a washing step, e.g., to remove the cryogenic protectant and/or DMSO. In some embodiments, standing is or includes culture and/or incubation at or about 37° C. In some embodiments, standing is carried out under any reagents (e.g., stimulatory reagents, bead reagents, or recombinant cytokines) used with the absence of and/or in combination with any treatment or engineering step. In some embodiments, the cells are allowed to stand for 5 minutes, 10 minutes, 15 minutes, 30 minutes, 1 hour, 2 hours, 3 hours, 4 hours, 5 hours, 6 hours, 8 hours, 1 hours, 18 hours, or 24 hours; or about 5 minutes, about 10 minutes, about 15 minutes, about 30 minutes, about 1 hour, about 2 hours, about 3 hours, about 4 hours, about 5 hours, about 6 hours, about 8 hours, about 12 hours, about 18 hours, or about 24 hours; or at least 5 minutes, at least 10 minutes, at least 15 minutes, at least 30 minutes, at least 1 hour, at least 2 hours, at least 3 hours, at least 4 hours, at least 5 hours, at least 6 hours, at least 8 hours, at least 12 hours, at least 18 hours, or at least 24 hours. In certain embodiments, the cells are allowed to stand for 2 hours, about 2 hours, or at least 2 hours.

In some embodiments, the percentage of viable cells is from 24% to 100% after the storage period. The percentage of viable cells may be determined, for example, by using AO/DAPI (acridine orange/4′,6-diamidino-2-phenylindole) fluorescent dye or trypan blue dye exclusion techniques. In one embodiment, according to principles of AO/DAPI fluorescent staining, AO (acridine orange) is able to penetrate intact cell membranes and be excited to generate green fluorescence signals to stain whole cells; while DAPI is able to penetrate only the permeabilized dead cell membranes and present blue fluorescence signals to stain dead cells; therefore, the green and blue fluorescence signals can be combined to determine the percentage of viable cells. In one embodiment, according to the trypan blue dye exclusion technique, for example, dead cells appear blue and are therefore distinguishable from viable cells. In one embodiment, the percentage of viable cells can also be determined, for example, by using a flow cytometer or another technique or instrument.

During the process of cooling, cryogenically freezing, and/or cryogenically storage of the sample or cells, one or more biological functions of the cells are retained. The use of a freezing medium helps retain these biological functions. These biological functions are restored when the cells are thawed. In addition to vitality (e.g., the biological functions described above), other biological functions may include the replication capacity of the cells, the acceptance of genetic modifications, and the ability to assist immune processes, including the maturation of B cells into plasma cells and/or memory B cells, and the activation of cytotoxic T cells and/or macrophages, etc.

In some embodiments, an increase and/or faster expansion of specific concentration or cell density of cells and/or the cell composition frozen and/or filled into a container with a specific volume or ratio of surface area to volume in the presence of a cryogenic protectant; increase and/or enhancement of cell viability, and decrease of cell death, such as necrosis, programmed cell death, and/or apoptosis; improvement, enhancement and/or increase of cytolytic activity; and; or reduction of aging or quiescence after thawing, after freezing the cells and/or the cell composition by alternative means.

In a particular embodiment, the cells are frozen at a cell density and; or ratio of surface area to volume provided herein, and the amount of cell death (e.g., necrosis and/or apoptosis) during or resulting from freezing, cryogenic freezing, and/or cryogenic storage is reduced, as compared to the cells frozen at a different cell density and/or ratio of surface area to volume under the same or similar conditions. In a particular embodiment, the cells are frozen at a cell density and/or ratio of surface area to volume provided herein, and within 48 hours after freezing, cryogenic freezing, and/or cryogenic storage, e.g., after thawing of the frozen cells, the amount of delayed cell death is reduced, e.g., the number of cells that die (e.g., via necrosis, programmed cell death, or apoptosis) is reduced. In certain embodiments, less than at least 5%, at least 10%, at least 20%, at least 25%, at least 30%, at least 40%, at least 50%, at least 60%, at least 70%, at least 80%, at least 90%, or at least 99%; or less than about 5%, about 10%, about 20%, about 25%, about 30%, about 40%, about 50%, about 60%, about 70%, about 80%, about 90%, or about 99% of the cells die during and/or die as a result of freezing and; or cryogenic storage, as compared to the cells frozen at a different cell density and/or ratio of surface area to volume under the same or similar conditions. In certain embodiments, less than 40%, 30%, 25%, 20%, 15%, 10%, 5%, 1%, 0.1%, or 0.01% of the cells frozen at the provided cell density and; or ratio of surface area to volume die during or die as a result of freezing, cryogenic freezing, and/or cryogenic storage.

In some embodiments, the cells are frozen at a cell density and/or ratio of surface area to volume provided herein, and the cases of aging or quiescence due to and/or resulting from freezing, cryogenic freezing, and/or cryogenic storage are reduced, as compared to the cells frozen at a different cell density and/or ratio of surface area to volume under the same or similar conditions. In a particular embodiment, less than at least 5%, at least 10%, at least 20%, at least 25%, at least 30%, at least 40%, at least 50%, at least 60%, at least 70%, at least 80%, at least 90%, or at least 99%; or less than about 5%, about 10%, about 20%, about 25%, about 30%, about 40%, about 50%, about 60%, about 70%, about 80%, about 90%, or about 99% of the cells are aging cells and/or quiescent cells, as compared to the cells frozen at a different cell density and/or ratio of surface area to volume under the same or similar conditions. In certain embodiments, the cells are frozen at a cell density and/or ratio of surface area to volume provided, and less than 40%, 30%, 25%, 20%, 15%, 10%, 5%, 1%, 0.1%, or 0.01% of the cells become aging and/or quiescent due to freezing, cryogenic freezing, and/or cryogenic storage.

In certain embodiments, the cells are frozen (e.g., cryogenically frozen) at a cell density and/or ratio of surface area to volume provided herein, and have improved, faster, and/or higher rate of expansion after thawing of the cells (e.g,, under stimulatory conditions, such as by incubation with a stimulatory reagent described herein), as compared to the cells frozen at a different cell density and/or ratio of surface area to volume under the same or similar conditions. In a particular embodiment, the cells are expanded at a rate of 5%, 10%, 20%, 25%, 30%, 40%, 50%, 60%, 70%, 80%, 90%, 100%, 150%. 200%, 1-fold, 1,5-fold, 2-fold, 3-fold, 4-fold, 5-fold, or 10-fold faster and/or higher; or about 5%, about 10%. about 20%, about 25%, about 30%, about 40%, about 50%, about 60%, about 70%, about 80%, about 90%, about 100%, about 150%, about 200%, about 1-fold, about 1.5-fold, about 2-fold, about 3-fold, about 4-fold, about 5-fold, or about 10-fold faster and/or higher; or at least 5%, at least 10%, at least 20%, at least 25%, at least 30%, at least 40%, at least 50%, at least 60%. at least 70%, at least 80%, at least 90%, at least 100%, at least 150%, at least 200%, at least 1-fold, at least 1.5-fold, at least 2-fold, at least 3-fold, at least 4-fold, at least 5-fold, or at least 10-fold faster and/or higher; as compared to the cells frozen at a different cell density and/or ratio of surface area to volume under the same or similar conditions. For example, in some embodiments, the thawed cells reach the threshold expansion (e.g., a predetermined cell number, density, or factor, such as 2-fold expansion) in 5%, 10%, 20%, 25%, 30%, 40%, 50%, 60%, 70%, 80%, 90%, 95%, or 99% less time; or about 5%, about 10%, about 20%, about 25%, about 30%, about 40%, about 50%, about 60%, about 70%, about 80%, about 90%, about 95%, or about 99% less time; or at least 5%. at least 10%, at least 20%, at least 25%, at least 30%, at least 40%, at least 50%, at least 60%, at least 70%, at least 80%, at least 90%, at least 95%, or at least 99% less time; as compared to the thawed cells frozen at a different cell density and/or ratio of surface area to volume under the same or similar conditions.

In some embodiments, the cells are frozen (e.g., cryogenically frozen) at a certain cell density, and have improved, increased, and/or greater cytolytic activity after thawing of the cells, as compared to the cells frozen at a different cell density (e.g., higher or lower density) under the same or similar conditions, for example, such as measured by any of the means for measuring cytolytic activity described herein. In a particular embodiment, the cytolytic activity is increased by 5%, 10%, 20%, 25%, 30%, 40%, 50%, 60%, 70%, 80%, 90%, 100%, 150%, 200%, 1-fold, 1.5-fold, 2-fold, 3-fold, 4-fold, 5-fold, or 10-fold; or about 5%, about 10%, about 20%, about 25%, about 30%. about 40%, about 50%, about 60%, about 70%, about 80%, about 90%, about 100%, about 150%, about 200%, about 1-fold, about 1.5-fold, about 2-fold, about 3-fold. about 4-fold, about 5-fold, or about 10-fold; or at least 5%, at least 10%, at least 20%, at least 25% at least 30%, at least 40%, at least 50%, at least 60%, at least 70%, at least 80%, at least 90%, at least 100%, at least 150%, at least 200%. at least 1-fold, at least 1.5-fold, at least 2-fold, at least 3-fold, at least 4-fold, at least 5-fold, or at least 10-fold; as compared to the cells frozen at a different cell density under the same or similar conditions.

In some embodiments, the genetic modification includes genetically modifying a cell to express one or more chimeric antigen receptors (CAR). Exemplary antigen receptors, including CAR, and methods for engineering such receptors and introducing such receptors into cells are, for example, those disclosed in Chinese Patent Application Publication Nos. CN107058354A, CN107460201A, CN105194661A, CN105315375A, CN105713881A, CN106146666A, CN106519037A, CN106554414A, CN105331585A, CN106397593A, CN106467573A, CN104140974A, CN108884459A, CN107893052A, CN108866003A, CN108853144A, CN109385403A, CN109385400A, CN109468279A, CN109503715A, CN109908176A, CN109880803A, CN110055275A, CN110123837A, CN110438082A, CN110468105A, CN109796532A; International Patent Application Publication Nos. WO2017186121A1, WO2018006882A1, WO2015172339A8, WO2018/018958A1, WO2014180306 A1, WO2015197016A1, WO2016008405A1, WO2016086813A1, WO2016150400A1, WO2017032293A1, WO2017080377A1, WO2017186121A1, WO2018045811A1, WO2018108106A1, WO2018/21.9299, WO2018/210279, WO2019/024933, WO2019/114751, WO2019/114762, WO2019/141270WO2019/149279, WO2019/170147A1, WO 2019/210863, WO2019/219029, WO2019047932A1, WO2020020210A1WO2020057666A WO2020057641A1, WO2020057668A1, WO2020/063988A1, WO2020/083406A1, WO2020114518A1, WO2020143631A1, WO2020156554A1, WO2020259707, WO2021/027785, WO2021/052496A1, WO2021/057906A1, and WO2018/13387. A chimeric receptor, such as CAR, typically comprises an extracellular antigen-binding domain, such as part of an antibody molecule, typically a variable heavy (VH) chain region (alternatively, referred to as a heavy chain variable region) and/or a variable light (VL) chain region (alternatively, referred to as a light chain variable region) of the antibody, such as a scFv antibody fragment. In some embodiments, the chimeric receptor comprises an extracellular antigen-binding domain that is not derived from an antibody molecule, such as a ligand or other binding moiety.

Preparations include those for oral, intravenous, intraperitoneal, subcutaneous, pulmonary, transdermal, intramuscular, intranasal, buccal, sublingual, or suppository administration. In some embodiments, the cell populations are administered parenterally. As used herein, the term “parenteral” includes intravenous, intramuscular, subcutaneous, rectal, vaginal, and intraperitoneal administration. In some embodiments, the cells are administered to a subject with peripheral system delivery by intravenous, intraperitoneal, or subcutaneous injection.

In some embodiments, the cell composition is provided as a sterile liquid preparation, such as an isotonic aqueous solution, suspension, emulsion, dispersion, or viscous composition, in some aspects, the preparation can be buffered to a selected pH. Liquid preparations are generally easier to prepare than gels, other viscous compositions, and solid compositions. In addition, liquid compositions are in a way more convenient for administration, especially by injection. In another aspect, the viscous compositions can be prepared within an appropriate viscosity range to provide an extended time of contact with a particular tissue. The liquid or viscous compositions may contain a carrier, which may be a solvent or dispersion medium including, for example, water, saline, phosphate buffered saline, polyols (for example, glycerol, propylene glycol, liquid polyethylene glycol), and suitable combinations thereof.

Sterile injectable solutions can be prepared by incorporating cells into a solvent, such as mixing with a suitable carrier, diluent, or excipient, e.g,, sterile water, normal saline, glucose, dextrose, and the like. Depending on the route of administration and the desired product, the composition may contain auxiliary substances, such as wetting agents, dispersants, or emulsifiers methylcellulose), pH buffers, gelling or viscosity enhancing additives, preservatives, flavoring agents, and/or coloring agents. In some aspects, a suitable product can be prepared with reference to standard text.

A variety of additives may be added to enhance the stability and sterility of the composition, including antimicrobial preservatives, antioxidants, chelating agents, and butlers. Prevention of action of microorganisms may be ensured by various antibacterial and antifungal agents, for example, parabens, chlorobutanol, phenol, and sorbic acid. Prolonged absorption of injectable drug forms may be achieved by the use of agents delaying absorption, such as aluminum monostearate and gelatin.

Preparations to be used for in vivo administration are typically sterile. Sterility may be readily achieved by, for example, filtration through sterile filter membranes.

In some embodiments, the therapeutic T cell composition comprises between about 10 million cells/mL and about 70 million cells/mL, or between about 10 million viable cells/mL and about 70 million viable cells/mL. In some embodiments, the therapeutic T cell composition comprises between about 15 million cells/mL or viable cells/int and about 60 million cells/mL or viable cells/mL. In some embodiments, the T cell composition comprises greater than 10 million cells/mL or viable cells/mL. In some embodiments, the therapeutic T cell composition comprises greater than 15 million cells/mL or viable cells/mL.

In some embodiments, the present application provides a product comprising a container containing a therapeutic T cell composition. In some embodiments, the product further comprises information indicating that the container contains a target number of units of the therapeutic T cell composition. In some embodiments, the product comprises a plurality of containers, wherein each of the containers comprises a unit dose comprising a target number of units of the T cell composition. In some embodiments, the container comprises between about 10 million cells/mL or viable cells/mL and about 70 million cells/mL, or viable cells/mL; or between about 15 million cells/mL or viable cells/mL and about 60 million cells/mL or viable cells/mL; or greater than 10 million cells/mL or viable cells/mL or greater than 15 million cells/mL or viable cells/mL or a combination thereof. In some embodiments, the composition further comprises a cryogenic protectant, and/or the product further comprises instructions for thawing the composition prior to administration to a subject.

The Advantages of the Present Application are as Follows:

In cell therapy, due to the limited number or viability of immune cells collected from donors (especially patients with advanced tumors), how to maximize the preservation of cell viability or functions is an important step in cell therapy. In particular, cell therapy for solid tumors often requires the infusion of large doses of highly viable immune cells into the subjects.

The present application has surprisingly found that reducing the concentration of DMSO, or simultaneously adding a low concentration of HSA, may significantly improve cell viability after recovery of the cells cryopreserved with the commercial cryopreservation medium CS5, and significantly reduce the decline of cell viability after resting at room temperature for 2 hours, 8 hours, or 24 hours after recovery reducing the concentration of DMSO, or simultaneously adding a low concentration of HSA, may significantly improve the expansion ability in vitro after recovery of the cells cryopreserved with the commercial cryopreservation medium CS5. The cryopreservation medium of the present application reduces cytotoxicity of high concentration of DMSO (for example, the side effects such as irritation of DMSO on blood vessels and oral mucosa, and metabolic burdens on organs such as liver and kidney) while improving recovery effects on cells after cryopreservation. A low concentration of USA provides both nutrients to the cells and non-permeable protective substances during cell cryopreservation, so as to better protect the cells to achieve higher viability and vitality.

The present application will now be further illustrated with reference to specific examples. It is understood that these examples are included merely for purposes of illustration, and are not intended to limit the scope of the application. Specific conditions of experimental methods are not specified in the following examples, which generally follow conventional conditions such as those described in Molecular Cloning: A Laboratory Manual, (J. Sambrook, et al. eds.) 3rd edition, Science Press. 2002, or conditions as suggested by manufacturers.

### EXAMPLE 1. COMPARISON OF CELL CRYOPRESERVATION EFFECTS OF COMMERCIAL CELL CRYOPRESERVATION MEDIA CRYOSTOR® CS2, CRYOSTOR® CS5, AND CRYOSTOR®CS10

Cells: CAR-T cells prepared from PBMCs of donors. The amino acid sequence of CAR is shown below:

(SEQ ID No: 1)
QVQLQQPGAELVRPGASVKLSCKASGYTFTSYWINW
VKQRPGQGLEWIGNIYPSDSYTNYNQKFKDKAILI
VDKSSSTAYMQLSSPTSEDSAVYYCTRSWRGNSFD
YWGQGTTLIVSSGGGGSGGGGSGGGGSDIVMTQSP
SSLTVTAGEKVTMSCKSSQSLLNSGNQKNYLTWYQ
QKPGQPPKLLIYWASTRESGVPDRFTGSGSGTDFT
LTISSVQAEDLAVYYCQNDYSYPFTFGSGTKLEIK
Rtttpaprpptpaptiasqplsirpeacrpaagga
vhtrgldfacdfwvlvvvggvlacysllvtvafii
fwvrskrsrilhsdymnmtprrpgptrkhyqpyap
prdfaayrsrvkfsrsadapayqqgqnqlynelnl
grreeydvldkrrgrdpemggkpqrrknpqeglyn
elqkdkmaeayseigmkgerrrgkghdglygglst
atkdtydalhmqalppr

The specific procedures for cryopreservation and recovery are as follows:

1. Cryopreservation: two batches of CAR-T cells respectively prepared by PBMCs from two donors were centrifuged, then removing the supernatant; CS2, CS5 and CS10 cryopreservation media were respectively added slowly to one part of the above cells to mix well, then adjusting the cell density to 5.00×10⁷ cells/mL. Each of the samples was distributed into three vials in the volume of 1 mL/vial. The vials were then cooled to −80 to −90° C. by a program-controlled cooling instrument at a rate of at least 1° C./min, and stored in liquid nitrogen; subsequently the samples were taken out for detection.

2. Recovery: three vials of each of the CAR-T cells cryopreserved with the three cryopreservation media above were respectively taken and immediately placed in a water bath at 38° C. tier rapid thawing; after complete thawing, the cell viability was determined by a cell counter and recorded as the cell viability at “0 h after recovery”. The percentage of viable cells may be determined, for example, by using AO/DAPI (acridine orange/4′,6-diamidino-2-phenylindole) fluorescent dye. The cells recovered above were allowed to stand at room temperature for 8 h, and the cell viability was determined by a cell counter and recorded as the cell viability at “8 h after recovery”.

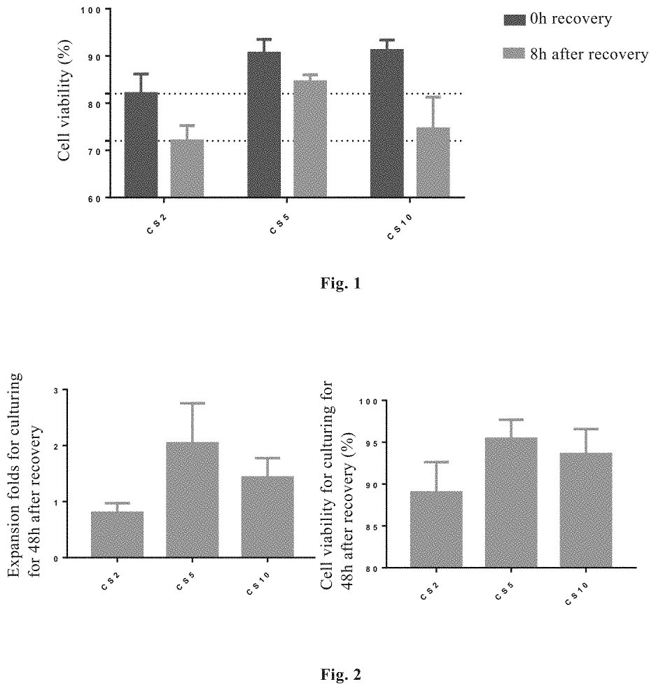

The results are shown in Table 1 below and FIG. 1. Except for CS2, the viability after recovery of CAR-T cells cryopreserved with cryopreservation media CS5 and CS10 is more than 90%. Compared with 0 h after recovery, the viability of CAR-T cells cryopreserved with the three cryopreservation media decreases at 8 h after recovery; and the cells cryopreserved with CS5 have the highest viability after recovery, and have the minimum decline of viability, suggesting that CS5 has the best protective effect on cells in cryopreservation.

TABLE 1
Cell viability of cells cryopreserved with cell cryopreservation
media CS2, CSS, and CS10 after recovery

| | Cell viability (%) | | | | | |
|---|---|---|---|---|---|---|
| Cryopreservation | 0 h after recovery | | | 8 h after recovery | | |
| medium formula | Donor 1 | Donor 2 | AVG | Donor 1 | Donor 2 | AVG |
| CS2 | 79.3 | 84.8 | 82.1 | 69.4 | 74.6 | 72.0 |
| CS5 | 88.2 | 92.2 | 90.2 | 83.0 | 85.6 | 84.3 |
| CS10 | 89.5 | 92.9 | 91.2 | 68.9 | 80.2 | 74.6 |

To simulate the limiting conditions for clinical use, the recovered samples were placed at room temperature for 2 h. then centrifuging to remove cryopreservation media. The cells were resuspended to 1.00×10⁶ cells/mL in AIM-V medium containing 2% human AB serum, inoculating into 6-well cell culture plates, 2 mL/well. After 48 h of culture at 37° C., 5% CO₂, the viable cell density and cell viability were detected by a cell counter, so as to calculate the expansion folds of CAR-T cells cryopreserved with different cryopreservation media after recovery and culture for 48 h. The results are shown in Table 2 below and FIG. 2. The cells cryopreserved with the cryopreservation medium CSS have an expansion folds of 1.96 and a culture viabilit of 95.0% after recovery and culture for 48 h, which are higher than those of the cells cryopreserved with CS2 and CS10. This suggests that CS5 cryopreservation medium has the best protective effect on cells in cryopreservation.

TABLE 2
Expansion folds and cell viability of cells cryopreserved with cell cryopreservation
media CS2, CS5, and CS10 after recovery and culture for 48 h

| | Total expansion folds after | | | Cell viability after recovery | | |
|---|---|---|---|---|---|---|
| Cryopreservation | recovery and culture for 48 h | | | and culture for 48 h (%) | | |
| medium formula | Donor 1 | Donor 2 | AVG | Donor 1 | Donor 2 | AVG |
| CS2 | 0.82 | 0.78 | 0.80 | 88.3 | 89.7 | 89.0 |
| CS5 | 1.54 | 2.37 | 1.96 | 93.1 | 97.0 | 95.0 |
| CS10 | 1.19 | 1.67 | 1.43 | 90.9 | 96.3 | 93.6 |

### EXAMPLE 2. COMPARISON OF CRYOPRESERVATION EFFECTS ON CELL FOR CELL CRYOPRESERVATION MEDIA WITH DIFFERENT CONCENTRATIONS OF DMSO AND HSAS

I. Considering that HSA has a certain protective effect on cell cryopreservation and recovery, it was further verified in this example that whether reducing the concentration of DMSO and adding low concentration of HSA on the basis of the existing cryopreservation media may improve the cell viability of cryopreserved cells after recovery. Researchers conducted the study by using the cryopreservation medium formulas as shown in Table 3.

TABLE 3
Cryopreservation medium formulas

| | Preparation proportion of | | | Final concentration | |
|---|---|---|---|---|---|
| | different agents (v/v) | | | after preparation | |
| Cryopreservation | | 20% | 5% | DMSO | HSA |
| medium formula | CS5 | HSA | HSA | (w/v) | (w/v) |
| Formula 1 | 9 | 1 | / | 4.50% | 2.00% |
| Formula 2 | 4 | 1 | / | 4.00% | 4.00% |
| Formula 3 | 3 | 1 | / | 3.75% | 5.00% |
| Formula 4 | 1 | / | 1 | 2.50% | 2.50% |
| CS5 | 1 | / | / | 5.00% | 0% |

Three batches of CAR-T cells were prepared by PBMCs from three donors. According to the cryopreservation and recovery methods in Example 1, the prepared CAR-T cells were cryopreserved with cryopreservation media of Formulas 1, 2, 3, 4, and CS5, respectively. The cell viability was observed and recorded at “0 h after recovery” and “8 h after recovery”. The results are shown in Table 4 below and FIG. 3.

TABLE 4
Cell viability of cells cryopreserved with different cryopreservation
media at 0 h after recovery and 8 h after recovery

| | Cell viability (%) | | | | | | | |
|---|---|---|---|---|---|---|---|---|
| Cryopreservation | 0 h after recovery | | | | 8 h after recovery | | | |
| medium formula | Donor 3 | Donor 4 | Donor 5 | AVG | Donor 3 | Donor 4 | Donor 5 | AVG |
| Formula 1 | 89.3 | 85.8 | 90.1 | 88.4 | 80.9 | 81.6 | 87.9 | 83.5 |
| Formula 2 | 90.6 | 85.5 | 91.1 | 89.1 | 83.6 | 82.1 | 87.2 | 84.3 |
| Formula 3 | 90.2 | 85.0 | 90.0 | 88.4 | 85.9 | 75.6 | 83.3 | 81.6 |
| Formula 4 | 87.5 | 85.7 | 87.9 | 87.0 | 82.1 | 78.0 | 79.8 | 80.0 |
| CS5 | 89.7 | 83.5 | 89.7 | 87.6 | 84.4 | 76.7 | 82.9 | 81.3 |

The results showed that the cell viability of cells cryopreserved with cryopreservation media of Formula 1, Formula 2 and Formula 3 after recovery was slightly higher than that of the cells cryopreserved with CS5. After standing at room temperature for 8 h, the cell viability of cells cryopreserved with different cryopreservation media after recovery decreases to different degrees, particularly, the viability of cells cryopreserved with Formula 1 and Formula 2 decreases the least, with a viability significantly higher than that of the cells cryopreserved with CS5. This suggests that reducing the concentration of DMSO to 4.5% (w/v) or 4% (w/v), and adding low concentration of HSA to 2% (w/v) or 4% (w/v) in a cell cryopreservation medium, can not only reduce the safety risk caused by high concentration of DMSO, but also increase the cell viability of the cryopreserved cells after recovery.

II. Further observation was made on necessities of reducing the concentration of DMSO and adding HSA in the cryopreservation medium, as well as the reasonability of its concentration. Cryopreservation medium formulas are shown in Table 5 below.

TABLE 5
Cryopreservation medium formulas

| | Preparation proportion of | | | | Final concentration | |
|---|---|---|---|---|---|---|
| | different agents (v/v) | | | | after preparation | |
| Cryopreservation | | 20% | 5% | Normal | DMSO | HSA |
| medium formula | CS5 | HSA | HSA | saline | (w/v) | (w/v) |
| Formula 2 | 4 | 1 | / | / | 4.00% | 4.00% |
| Formula 5 | 8 | 1 | / | 1 | 4.00% | 2.00% |
| Formula 6 | 4 | / | / | 1 | 4.00% | 0% |
| Formula 7 | 3 | 1 | / | 1 | 3.00% | 4.00% |
| Formula 8 | 3 | / | 2 | / | 3.00% | 2.00% |
| Formula 9 | 3 | / | / | 2 | 3.00% | 0% |
| Formula 10 | 2 | 1 | / | 2 | 2.00% | 4.00% |
| Formula 11 | 2 | / | 2 | 1 | 2.00% | 2.00% |
| Formula 12 | 2 | / | / | 3 | 2.00% | 0% |

(1) Cell Viability of Cells Cryopreserved with Different Cryopreservation Media After Recovery

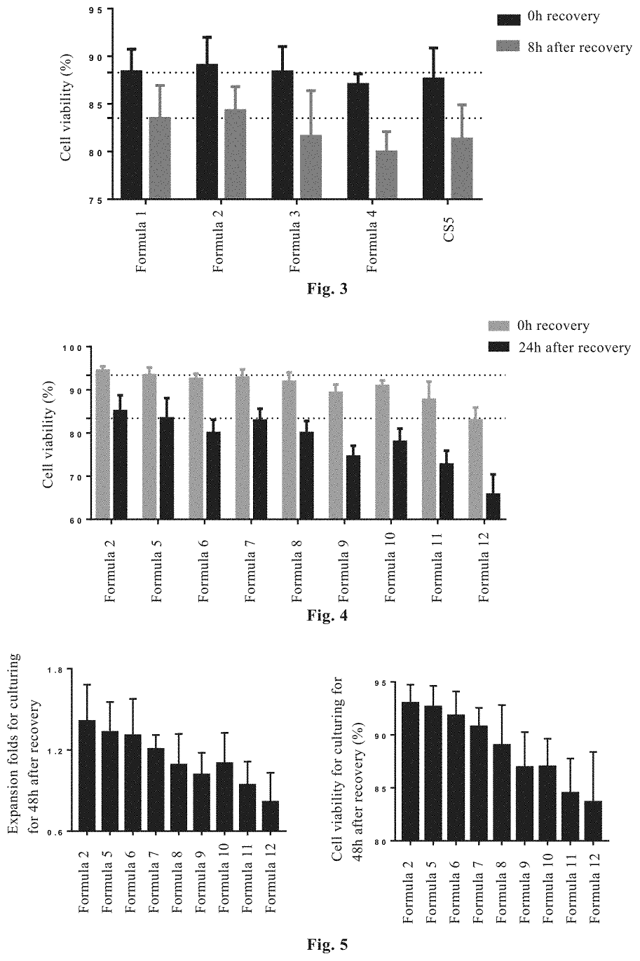

Three batches of CAR-T cells were prepared by PBMCs from three donors. According to the cryopreservation and recovery methods in Example 1, the prepared CAR-T cells were cryopreserved with cryopreservation media of Formulas 2, 5, 6, 7, 8, 9, 10, 11 and 12, respectively. The cell viability was observed and recorded at “0 h after recovery”. In order to fully expose toxic effects of the cryopreservation media on cells, the cells cryopreserved with the above cryopreservation media were allowed to stand at room temperature for 24 h after recovery, and the cell viability was determined by a cell counter and recorded as the cell viability at “24 h after recovery”, The results are shown in Table 6 below and FIG. 4.

TABLE 6
Cell viability of cells cryopreserved with different cryopreservation
media at 0 h after recovery and 24 h after recovery

| | Cell viability after recovery (%) | | | | | | | |
|---|---|---|---|---|---|---|---|---|
| Cryopreservation | 0 h after recovery | | | | 24 h after recovery | | | |
| medium formula | Donor 3 | Donor 4 | Donor 5 | AVG | Donor 3 | Donor 4 | Donor 5 | AVG |
| Formula 2 | 95.3 | 94.1 | 94.1 | 94.5 | 88.4 | 80.7 | 86.2 | 85.1 |
| Formula 5 | 95.5 | 92.5 | 92.3 | 93.4 | 86.9 | 77.4 | 85.9 | 83.4 |
| Formula 6 | 93.9 | 91.7 | 92.1 | 92.6 | 83.2 | 76.9 | 79.9 | 80.0 |
| Formula 7 | 94.7 | 92.8 | 91.1 | 92.9 | 84.5 | 79.7 | 84.4 | 82.9 |
| Formula 8 | 94.3 | 90.4 | 90.9 | 91.9 | 81.3 | 76.9 | 81.8 | 80.0 |
| Formula 9 | 91.1 | 87.5 | 89.3 | 89.3 | 73.7 | 72.7 | 77.3 | 74.6 |
| Formula 10 | 91.6 | 90.3 | 90.8 | 90.9 | 78.4 | 75.1 | 80.4 | 78.0 |
| Formula 11 | 91.3 | 88.6 | 83.2 | 87.7 | 75.4 | 71.7 | 71.0 | 72.7 |
| Formula 12 | 85.2 | 81.2 | 82.4 | 82.9 | 67.3 | 60.5 | 69.5 | 65.8 |

(2) Expansion Folds of Cells Cryopreserved with Different Cryopreservation Media After Recovery and Culture for 48 h

After the recovered samples were centrifuged to remove cryopreservation media, the cells were resuspended to 1.00×10⁶ cells/mL in AIM-V medium containing 2% AB serum, then inoculating into 6-well cell culture plates, 2 mL/well. After 48 h of culture at 37° C., 5% CO₂, the viable cell density and cell viability were detected by a cell counter, so as to calculate the expansion folds of CAR-T cells cryopreserved with different cryopreservation media after recovery and. culture for 48 h. The results are shown in Tables 7 and 8 below and FIG. 5.

TABLE 7
Expansion folds of cells cryopreserved with different cryopreservation
media after recovery and culture for 48 h

| | |
|---|---|
| | Total expansion folds after |
| Cryopreservation | recovery and culture for 48 h |

| medium formula | Donor 3 | Donor 4 | Donor 5 | AVG |
|---|---|---|---|---|
| Formula 2 | 1.67 | 1.30 | 1.28 | 1.42 |
| Formula 5 | 1.42 | 1.35 | 1.22 | 1.33 |
| Formula 6 | 1.20 | 1.38 | 1.36 | 1.31 |
| Formula 7 | 1.27 | 1.16 | 1.20 | 1.21 |
| Formula 8 | 0.98 | 1.19 | 1.11 | 1.09 |
| Formula 9 | 1.04 | 1.16 | 0.86 | 1.02 |
| Formula 10 | 1.32 | 1.11 | 0.88 | 1.10 |
| Formula 11 | 1.08 | 0.90 | 0.84 | 0.94 |
| Formula 12 | 0.73 | 1.09 | 0.64 | 0.82 |

TABLE 8
Cell viability of cells cryopreserved with different cryopreservation
media after recovery and culture for 48 h

| | |
|---|---|
| | Cell viability after |
| Cryopreservation | recovery and culture for 48 h (%) |

| medium formula | Donor 3 | Donor 4 | Donor 5 | AVG |
|---|---|---|---|---|
| Formula 2 | 94.4 | 93.4 | 91.1 | 93.0 |
| Formula 5 | 94.0 | 92.7 | 91.2 | 92.6 |
| Formula 6 | 91.4 | 92.4 | 91.6 | 91.8 |
| Formula 7 | 90.6 | 91.0 | 90.8 | 90.8 |
| Formula 8 | 88.6 | 90.8 | 87.7 | 89.0 |
| Formula 9 | 87.6 | 89.0 | 84.2 | 86.9 |
| Formula 10 | 89.0 | 87.3 | 84.6 | 87.0 |
| Formula 11 | 86.0 | 86.0 | 81.4 | 84.5 |
| Formula 12 | 80.6 | 89.3 | 81.1 | 83.7 |

The results above show that the expansion folds and cell viability of the cells cryopreserved with different cryopreservation media after recovery and culture for 48 h tend to decrease with the decrease of concentrations of DMSO and HSA in the cryopreservation medium formulas.

All documents mentioned in the present application are incorporated herein by reference as if each of the documents were individually incorporated by reference. Moreover, it is understood that after reading above teachings of the present application, those skilled in the art may make various changes and modifications to the present application, and such equivalents also fall within the scope of the appended claims.

1. A cryopreservation medium for a biological sample, comprising cryoprotectant and cryopreservation medium base solution, wherein the cryoprotectant comprises a combination of one or more of dimethyl sulfoxide (DMSO), glycerol and ethylene glycol, and the cryoprotectant has a concentration of about 1.0% to 6.0% (w/v) in the cryopreservation medium for a biological sample.

2. The cryopreservation medium according to claim 1, wherein the cryoprotectant is DMSO and has a concentration of about 2.0% to 6.0%, or about 2.0% to 4.5%, or about 2.0% to 4.0%, or about 2.0% to 3.8%, or about 2.0% to 3.75%, or about 2.0% to 3.0%, or about 2.5% to 4.5%, or about 2.5% to 4.0%, or about 2.5% to 3.8%, or about 2.5% to 3.75%, or about 2.5% to 3.0%, or about 3.0% to 4.5%, or about 3.0% to 4.0%, or about 3.0% to 3.8%, or about 3.0% to 3.75%, or about 3.75% to 4.5%, or about 3.8% to 4.5%, or about 3.75% to 4.0%, or about 3.8% to 4.0%, or about 4.0% to 4.5% (w/v) in the cryopreservation medium for a biological sample.

3. The cryopreservation medium according to claim 2, further comprising HSA; wherein the HSA has a concentration of about 1.0% to 6.0%, or about 2.0% to 5.0%, or about 2.0% to 4.0%, or about 2.5% to 5.0%, or about 2.5% to 4.0%, or about 4.0% to 5.0% (w/v) in the cryopreservation medium for a biological sample;
preferably, the HAS comprises recombinant human albumin and/or human serum albumin;
more preferably, the HAS is human serum albumin.

4. The cryopreservation medium according to any one of claims 1 3 claim 3, wherein in the cryopreservation medium for a biological sample, the DMSO has a concentration of about 2.0%, or about 3.0%, or about 3.8%, or about 3.75%, or about 4.0%, or about 4.5% (w/v); and/or the HSA has a concentration of about 2.0%, about 2.5%, about 4.0%, or about 5.0% (w/v).

5. The cryopreservation medium according to claim 3, wherein in the cryopreservation medium for a biological sample,
the DMSO has a concentration of about 2.0% (w/v), and the HSA has a concentration of about 2.0% (w/v); or
the DMSO has a concentration of about 2.5% (w/v), and the HSA has a concentration of about 2.5% (w/v); or
the DMSO has a concentration of about 3.0% (w/v), and the HSA has a concentration of about 2.0% (w/v); or
the DMSO has a concentration of about 3.0% (w/v), and the HSA has a concentration of about 4.0% (w/v); or
the DMSO has a concentration of about 4.0% (w/v), and the HSA has a concentration of about 2.0% (w/v); or
the DMSO has a concentration of about 4.5% (w/v), and the HSA has a concentration of about 2.0% (w/v); or
the DMSO has a concentration of about 4.0% (w/v), and the HSA has a concentration of about 4.0% (w/v); or
the DMSO has a concentration of about 3.8% (w/v), and the HSA has a concentration of about 5.0% (w/v); or
the DMSO has a concentration of about 3.75% (w/v), and the HSA has a concentration of about 5.0% (w/v).

6. The cryopreservation medium according to claim 1, wherein the cryopreservation medium base solution is any one, two, or three selected from the group consisting of phosphate buffered saline (PBS), CryoStor® CS5, CryoStor® CS2 and CryoStor® CS10, or any combination thereof

7. The cryopreservation medium according to claim 6, wherein the cryopreservation medium for a biological sample is prepared by adjusting the concentration of the cryoprotectant in the cryopreservation medium base solution to an extent that the cryoprotectant has a concentration of about 1.0% to 6.0% (w/v) in the cryopreservation medium for a biological sample.

8. The cryopreservation medium according to claim 7, wherein the cryopreservation medium for a biological sample is prepared by adjusting the concentrations of the cryoprotectant and the human serum albumin (HSA) in the cryopreservation medium base solution to an extent that the cryoprotectant has a concentration of about 1.0% to 6.0% (w/v), and the human serum albumin (HSA) has a concentration of about 1.0% to 6.0% in the cryopreservation medium for a biological sample.

9. The cryopreservation medium according to claim 3, wherein the HSA is any one selected from plasma extracted human albumin and genetically recombinant human albumin, or a combination thereof.

10. The cryopreservation medium according to claim 1, wherein the biological sample is or is derived from an apheresis sample, optionally a leukapheresis sample; and/or wherein the sample comprises leukocytes and/or lymphocytes, and/or wherein cells or blood cells in the sample essentially consist of leukocytes, or wherein at least 80%, at least 85%, at least 90%, at least 95%, at least 96%, at least 97%, at least 98%, or at least 99% of the cells in the sample, or at least 80%, at least 85%, at least 90%, at least 95%, at least 96%, at least 97%, at least 98%, or at least 99% of the blood cells in the sample are leukocytes.

11. The cryopreservation medium according to claim 1, wherein the biological sample is stored for a period, and wherein after the period the percentage of viable cells in the biological sample is about 24% to about 100%, or at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%.

12. The cryopreservation medium according to claim 1, wherein the biological sample comprises T cells or engineered T cells;
preferably, the biological sample is enriched, and comprises CD4⁺T cells or sub-populations thereof and/or CD8⁺T cells or sub-populations thereof; preferably, the T cells are primary T cells;
preferably, the T cells are derived from an autologous or allogeneic source;
preferably, the engineered T cells comprise T cells expressing a recombinant molecule or exogenous molecule;
preferably, the recombinant molecule or exogenous molecule is optionally a recombinant protein, or optionally a recombinant receptor which is optionally a T cell receptor (TCR), a chimeric receptor, a chimeric antigen receptor (CAR), or a combination thereof.

13. A cell composition, comprising:
cells, and
the cryopreservation medium according to claim 1; wherein
preferably, the cells are immune cells, mesenchymal stem cells, or a combination thereof,
preferably, the cells are peripheral blood mononuclear cell-derived cells.

14. The cell composition according to claim 13, wherein the immune cells comprise, but are not limited to: mononuclear cells, NK cells, B cells and T cells; preferably, the T cells comprise, but are not limited to: LAK, TIL, CIK, CTL, CAR-T and TCR-T;
preferably, the immune cells comprise T cells or engineered T cells;
preferably, the immune cells are enriched, and comprises CD4⁺T cells or sub-populations thereof and/or CD8⁺T cells or sub-populations thereof;
preferably, the T cells are primary T cells;
preferably, the T cells are derived from an autologous or allogeneic source;
preferably, the engineered T cells comprise T cells expressing a recombinant molecule or exogenous molecule;
preferably, the recombinant molecule or exogenous molecule is optionally a recombinant protein, or optionally a recombinant receptor which is optionally a T cell receptor (TCR), a chimeric receptor, a chimeric antigen receptor (CAR), or a combination thereof.

15. A method for cryopreservation of cells, comprising the following steps:
(i) mixing cells to be cryopreserved with the cryopreservation medium according to claim 1 to obtain a cell composition; and
(ii) cooling the cell composition obtained in the above step (i), then placing in a container at about −80° C. to −90° C. or in the gas phase of liquid nitrogen, wherein the container is optionally a bag or a vial.

16. The method according to claim 15, wherein the cells to be cryopreserved are cooled under programmed control at a rate of, or about, or greater than 1° C./min, optionally until the temperature reaches about −80° C. to −90° C.
